(19) United States
(12) Patent Application Publication
(10) Pub. No.: US 2024/0132568 A1
(43) Pub. Date: Apr. 25, 2024

(54) ALLOSTERIC COUPLING OF ANTIBODY AND NATURALLY SWITCHABLE, MULTI-SUBUNIT OUTPUT PROTEIN

(71) Applicant: NovaPro, Inc., Melbourne, FL (US)

(72) Inventor: Iain Peters, Melbourne, FL (US)

(73) Assignee: NovaPro, Inc., Melbourne, FL (US)

(21) Appl. No.: 18/494,524

(22) Filed: Oct. 24, 2023

Related U.S. Application Data
(60) Provisional application No. 63/380,870, filed on Oct. 25, 2022.

(51) Int. Cl. C07K 14/705; C07K 16/28; G01N 33/542

(52) U.S. Cl. CPC C07K 14/705; C07K 16/28; G01N 33/542; C07K 2317/35; C07K 2319/00

(57) ABSTRACT

A system for responding to the presence of an antigen or hapten analyte including: (1) an allosteric multi-subunit protein having a first conformational state associated with a first biological activity and a second conformational state associated with a second biological activity; and (2) an antibody including: (a) a heavy chain variable domain subunit of the allosteric protein fused to a first subunit of the allosteric multi-subunit protein, and (b) a light chain variable domain subunit of the allosteric protein fused to a second subunit of the allosteric multi-subunit protein; wherein the first biological activity is observable when the antigen or hapten analyte is bound to the heavy chain variable domain subunit and the light chain variable domain subunit; and wherein the second biological activity is observable when the antigen or hapten analyte is not bound to the heavy chain variable domain subunit and the light chain variable domain subunit.

### RELATED APPLICATIONS

This application claims priority under 35 U.S.C. § 119(e) of U.S. Provisional Patent Application Ser. No. 63/380,870 (Attorney Docket No. 4581.00007) filed on Oct. 25, 2022 and titled ALLOSTERIC COUPLING OF ANTIBODY AND NATURALLY SWITCHABLE, MULTI-SUBUNIT OUTPUT PROTEIN. The content of this application is incorporated herein by reference.

### FIELD OF THE INVENTION

The present invention relates to systems and methods for

### BACKGROUND OF THE INVENTION

IF-THEN protein devices are composed of 1-3 polypeptides working in concert to provide a particular output (THEN) in response to presence of the input (IF) stimulus. These molecular ‘machines’ have been the subject of intense research, particularly over the past decade, and have a wide range of exciting applications. They show great promise as biosensors in medicine, particularly one-step homogeneous assays for point-of-care (PoC) and personalized healthcare. In such an instance, the ‘input’ would be the presence of pathogen, anti-pathogen Ab or disease biomarker, whatever the assay is to detect, and the output would be a readable signal, be it a change in fluorescence or bioluminescence intensity/color, or enzyme activity measured with colorimetric substrate or by bioelectrochemistry. A second major category of use is in cellular studies where concentrations and even subcellular locations of specific metabolites and messengers are monitored (input; IF) via in vivo expression of proteins engineered to give fluorescent/bioluminescent tagging and quantitation (output; THEN) in real time, in cultured cells and even living animals. Similarly, with equally amazing insights into cell biology, many such devices are built to respond to input of light of a specific wavelength (and high spatial precision) provided by scientists to mediate activation/inactivation (output) of enzymatic or binding activity of a specifically chosen cell signaling mediator for cytoskeletal remodeling or other cellular function. Moreover, engineered IF-THEN systems are making a huge impact on advanced biotherapeutics for medicine: consider the role that CAR T-cells are playing in the fight against cancer, albeit using a cellular-based switch that technically falls outside the definition given above. This therapy is largely based on the T-cell response to formation of an intercellular surface complex approximating the ‘immune synapse’; antibody specific for cancer cell surface biomarker replaces the TCR receptor domain and allows the T-cell activation to be triggered by cell-to-cell binding, via this antibody, to the targeted cancer.

Devices with protease IF inputs are also useful. These types of protein switches have been used to create protease cascades to amplify output signal, as well as for synthetic biology. Biosensors for specific proteases have been constructed based on pseudo duplication of a segment of the output protein such that its cleavage by the protease analyte (input) releases the duplicated segment, allowing its replacement to insert and either restore barnase activity for HIV-1 protease detection or change FP from green to yellow emission in response to thrombin processing. Additionally, biosensors for a range of viral and regulatory cascade proteases have been made using linker cleavage to control proximity, and thus emission color, of a FRET pair of fluorescent proteins as well as bioluminescence intensity by altering relative orientation for Firefly luciferase N and C-terminal domains. Finally, viral protease inhibitor drugs were used to control covalent integrity of polyproteins for cellular studies by inhibiting self-processing proteolytic activity of hepatitis C NS3 that would otherwise autocatalytically cleave its fusion to flanking protein domains. This was used to control gene expression by either releasing transcription factor from a plasma membrane anchor or breaking linkage between trans-activation and DNA-binding domains.

Prior art assembly-mediated is a style of IF-THEN device engineering founded on using molecular pairings between the receptor and its target (or the disruption thereof), to change the relative positioning of output module elements. Often, the assembly will bring a fluorescent protein or dye in proximity to a spectrally overlapping donating fluorophore or light-emitting luciferase enzyme, for output signal arising from changes in FRET/BRET efficiency. Another common output regime for this type of design is through reconstitution of a luciferase, or other output module, by bringing protein fragments of the split enzyme together so they can refold by association to regain (at least partial) activity. In addition, these designs can be used to stipulate a particular cellular protein substrate, or subcellular location, as being targeted by the construct, under specific input conditions (eg illumination or chemical-inducer). It is worth noting that most biosensors currently available in the commercial healthcare sector are in fact based on the assembly-mediated design paradigm. Assembly-based IF-THEN protein designs can be further categorized as outlined below.

A) Assembly Controls Proximity of Output Entities Not Tethered to One Another

The first examples of this style of IF-THEN protein device engineering utilize what is commonly referred to as a ‘sandwich’ of two distinct antibodies or other receptors bound simultaneously at non-overlapping epitopes on a single molecule of target analyte FIG. 3. Such a sandwich format homogenous assay that can be adapted to any appropriate pair of Ab's without need for gene construction has been developed. This assay is based on protein G directed photoconjugation of complementary split nanoLuc protein fragments to constant regions of a pair of commercially available purified Ab's, with their assembly on target analyte resulting in reconstitution of nanoLuc and bioluminescent detection. Similar designs are based on genetic fusion of non-overlapping scFv's with split b-galactosidase, firefly luciferase or DNA-binding and trans-activation domains for gene expression output. Meanwhile, by using DNA ‘adaptors’ to build a multilayer assembly that ultimately directs association of split firefly luciferase fragments, any desired RNA target can be detected through the reconstituted bioluminescence activity. Target sequence specificity is through base-pair hybridization of single-stranded tails of the DNA adapters to adjacent sites on the RNA, with subsequent pairing of luciferase fragments mediated via binding of the fused zinc-finger protein domains to double-stranded regions of these adaptors. Alternatively, a biosensor for the PIP3 lipid second messenger has been created by fusing PIP3-binding pleckstrin homology domains to the C-terminal domain of firefly luciferase while its N-terminal segment was localized to the plasma membrane via an anchor-signal peptide. Recruitment of the pleckstrin-C domain fusion to bind PIP3 in the membrane leads to colocalization of the luciferase fragments and ensuing reconstitution of bioluminescence activity, which was detected via BRET from it to a spectrally overlapping fluorescent protein (FP) also fused to the PIP3-binding module. Conversely, assembly of a sandwich complex of non-overlapping Ab's on an antigen target molecule has been used to detect the latter by preventing gramicidin fused to one Ab from moving freely in an artificial lipid bilayer due to covalent attachment of the other Ab to the underlying support surface. Gramicidin must dimerize to form ion-conducting channels, and thus antigen-mediated curtailment of Ab-gramicidin migration is detected by reduced electrical conductivity through the membrane due inability of this protein to pair with gramicidin monomers (not fused to Ab) that are immobilized as well.

A similar strategy utilizes assembly on identical binding sites of full-length, i.e. bivalent, antibody to detect it as analyte, through simultaneous binding of epitope/antigen polypeptides fused separately to complementary split enzyme fragments (FIG. 4). In one case, identical peptide epitopes were fused to a short transmembrane (TM) domain (from EGFR) with a dimer of b-glucuronidase (GUS) subunits attached to the opposite termini of each, and these fusion polypeptides integrated into protocell vesicles by expressing in an in vitro transcription/translation system. Spontaneous insertion of the fusion polypeptides into vesicle membranes results in surface display of the epitope peptides such that binding to the bivalent Ab analyte causes pairing of the GUS dimers to form active tetramer within the protocell lumen. Ensuing activation of the b-glucuronidase (GUS) leads to accumulation of fluorescent product of catalysis that is commensurate with Ab binding, allowing a strong signal to be developed during incubation period of the assay. Moreover, this device was made modular for facile conversion to alternate target specificity by fusing the epitope peptide (or other receptor for bivalent analyte) of choice to the SpyCatcher adapter domain and then, in a spontaneous reaction, covalently conjugate it to SpyTag peptide fused to the TM-GUS construct. Meanwhile, polyclonal Ab against SARS-CoV-2 was detected via their bivalent capture of engineered proteins that carry complementary fragments of nanoLuc separately fused to identical highly immunogenic protein domains from the virus; long linkers used for these fusions allow the split luciferase fragments captured on the two arms of the same Ab to pair (complementary 50% of the time) and therein reconstitute bioluminescence activity.

‘Two-hybrid’ systems based on reconstitution of split enzymes fused to cellular proteins have also been developed. Conventional two-hybrid systems are generally not self-contained, relying on downstream events such as up-regulation of FP gene expression. However, the split enzyme systems are not dependent upon cellular processing to generate detection signal and furthermore will pinpoint position of complex formation within the cell if based on a light-emitting protein. For example, split luciferase fragments fused to a variety of protein-protein interaction (PPI) partners was used to monitor their association through bioluminescence; PPIs studied this way include GPCR with b-arrestin, Myc and Notch with partner transcription inducers, homo vs. heterodimerization of EGFR, as well as characterization of 132 primary interactions of 12 auxin proteins with 8 response factors in Arabidopsis. In addition, a FRET-based two-hybrid system was developed by labeling interacting proteins with spectrally overlapping FP (FIG. 5) to aid screening of anti-cancer drug candidates meant to block a particular PPI. Meanwhile, a two-color bioluminescence two-hybrid construct was developed to distinguish hetero and homodimers of the a and b-estrogen receptors (ER) by fusing the former to both N-domain of firefly and C-domain of Renilla luciferases and co-express with b-ER fused to C-domain of firefly and a second copy of a-ER, fused to N-domain of Renilla luciferases. Additionally, split luciferase fusion to Apaf-1 protomers of the homoheptameric apoptosome has been used to monitor assembly of this supramolecular complex central to initiation of cell death.

B) Bivalent Binding to Duplicated Receptors Embedded within Linker Between Output Modules

The basic concept here is that by placing two identical peptide epitopes within the linker that connects a pair of interacting protein entities, signal for presence of the epitope-specific Ab can be generated through its bivalent binding to force extension of the linker with concomitant separation of the output modules (FIG. 6). A semi-rigid linker composed of two 45 Å a-helices interspersed with flexible Gly-rich segments has been found to be optimal for efficiently spanning distance between Ab arms, without reducing baseline interaction of protein modules due to excessive flexibility [41]. An early example of this approach utilized a heterodimerizing FRET-donor/acceptor pair of FPs (engineered from non-interacting CyPet and YPet) to provide Ab detection through disrupted dimerization via linker extension that reduced FRET efficiency. More recently, a similar design used an accessory dimerizing domain to pair nanoLuc and acceptor FP for a BRET signal that is reduced through Ab binding to the duplicated epitope linker that connects them. This protein sensor has been incorporated into a microfluidic thread-based analytical device for multiplexed detection of several Ab's in a drop of blood within about 5 minutes using a digital camera. Other designs have been based on an increase in b-lactamase activity resulting from Ab-induced separation of it from its natural protein inhibitor, BLIP. After carefully balancing relative stabilities of the enzyme/inhibitor and Ab/epitope complexes, and using a fluorescent lactamase substrate, a roughly 10-fold increase in signal with a variety of monoclonal Abs, including against HIV, Dengue and Influenza viruses has been achieved. This b-lactamase/BLIP design has been extended to the detection of other multi-subunit proteins, by replacing epitope peptides with affimers, which are an antibody mimetic based on the cystatin protein scaffold. Not surprisingly, detection of anti-cancer Ab, Herceptin, has been best done using a semi-rigid linker based on that described above, however constructs for human C-reactive protein, a pentameric biomarker for acute phase inflammation, and the CPMV plant virus particle performed best when duplicated affimers were tethered using an entirely flexible (Gly-Ser-Gly)7 linker. Although these sensors were based on binding to repeated sites on the multimeric targets, it would also be effective for non-overlapping binding sites recognized by distinct affimers, so long as the distance that separates them is sufficient. Since these sensor proteins are single-chain, they avoid complications resulting from concentration-dependence of two-component systems, while using stably folded output modules prevents instability often observed for split enzyme designs. Furthermore, simultaneous binding of the target to two linked epitopes increases its apparent affinity for the sensor by up to 2-orders of magnitude over that of the monomeric binding reaction.

C) Monovalent Receptor-Analyte Binding Causes Displacement or Condensation

Simple monovalent interaction of a linker-embedded peptide ligand or binding domain with its target analyte protein, can effectively tighten the linker due to extra bulk of the bound protein and/or condensation of the linker-embedded element to accommodate rigid structural requirements of the complex. For detection of monoclonal Abs specific for HA and Myc peptide-tags, the corresponding epitopes were introduced into a short linker connecting b-lactamase with its BLIP protein inhibitor (FIG. 7). Ab-binding effectively tightened the linker, pulling BLIP away from its binding site and resulting in a modest increase in lactamase activity. A similar mechanism has been used to detect interaction of the barstar inhibitor with barnase RNAse protein incorporated into the linker connecting the t-FhuA nanochannel to a negatively charged blocking peptide. Binding of barstar causes the blocking peptide to be pulled back from the nanochannel pore and the resulting increase in ion-conduction was measured electrically using just a single molecule of the nanopore protein inserted within the phospholipid membrane barrier.

Alternatively, analyte binding to its receptor within the biosensor protein can be used to displace a chemical entity otherwise retained in or near the binding pocket. Release of this chemical entity by competing analyte binding leads to the output signal, either directly through changes to its optical properties or via modification to the biosensor protein configuration (FIG. 8). Q-bodies, in which a tethered fluoroscent dye (TAMRA) that has affinity for Trp residues commonly found in the binding sites of Abs, provide detection signal for target analyte binding through enhanced fluorescence of the displaced dye measured directly or via BRET from luciferase enzyme fused to the Ab. The latter approach avoids technical complications of providing effective illumination from an external source for optical excitation of the TAMRA fluorophore. This design paradigm has also been extended to Ab without Trp-rich combining sites by using combinatorial yeast display to identify TAMRA-peptide conjugates that, in the first phase of selection, bind to the Ab and then, in the second, are displaced by target analyte. Disruption of a silencing complex with the output module due to competitive binding from the target analyte, is also the basis of the Cloned Enzyme Donor ImmunoAssay (CEDIA). This detection assay has a split enzyme format founded on the well-studied a-peptide complementation of b-galactosidase activity and has been successfully marketed for monitoring drug dosage in patient sera. Assay setup has the a-complementation peptide sequestered in complex with the analyte-targeting Ab due to its conjugation to appropriate hapten; competition from analyte drug in the patient sample displaces the conjugate and allows the a-peptide to restore the homotetrameric structure and activity of b-galactosidase by being incorporated into the dimer-dimer interface. Furthermore, this blood test has been adapted to a microfluidic format that consumes a much smaller volume of patient sample, while being substantially faster and easier use than the conventional liquid assay. In another single-step biosensor device for PoC therapeutic drug monitoring, nanoLuc luciferase is genetically fused via a short linker, to the N-terminus of the drug-binding Fab antibody input receptor, while the other nanoLuc terminus is fused to the SNAP-tag protein domain through a rigid poly-Pro linker. This fusion protein is then modified at the SNAP-tag domain by covalently coupling it to a benzylguanine derivative that contains fluorescent dye tethered to a Fab-binding ligand related to the hapten. In the absence of drug analyte, binding between the Fab and the SNAP-conjugated hapten-like ligand creates a circularized polyprotein that has nanoLuc positioned close to the acceptor fluorophore dye (tethered to Fab-ligand), resulting in an efficient BRET signal. Conversely, drug present in analyzed samples displaces the conjugated hapten-like ligand from its binding to the Fab, resulting in a substantially increased separation between nanoLuc and the BRET-acceptor fluorophore dye (aided by the stiff Pro-linker between nanoLuc and SNAP). This causes an analyte-induced reduction in BRET, providing a ratiometric signal that can be used to accurately quantitate drug concentration through digital photography of filter paper impregnated with this biosensor plus luciferase substrates, and spotted with a small volume of patient serum (or 10-fold diluted blood) in parallel with known drug standard for calibration. Given the high effective local concentration of the tethered Fab ligand, it is essential that its affinity be sufficiently low to ensure displacement by a useful response range of drug analyte. Early designs also intended for PoC applications were similar except luciferase was replaced with FP so that detection is via a reduction in FRET efficiency. Meanwhile, an extension of this approach uses linear fusion of neuroreceptor with two tandem self-labeling protein tags (CLIP and SNAP) to facilitate creation of semi-synthetic biosensor constructs directly on the surface of living cells. Covalent adducts to the self-labeling tags are a pair of spectrally overlapping fluorescent dyes, that in the distal case is also tethered to ligand for the agonist-binding site of the neuroreceptor. Again, without neurotransmitter present this biosensor construct doubles back on itself to insert the tethered agonist analogue ligand into the receptor binding site; this yields strong FRET signal, while the open sensor configuration resulting from neurotransmitter-mediated release of tethered ligand, does not.

D) Power of Competition in the Fragment Pairing Reaction

Recent work has revealed the utility of an input-induced shift in competition between alternate partners for the reconstitution of complementary fragments as a way to reduce background activity and improve signal-to-noise (S/N) ratio of split protein-based assays. Albumin protein present at high concentrations in the blood serum samples, has been bound to the LgBiT luciferase fragment and therein blocked its association with the SmBiT peptide tethered to its natural N-terminus as part of a single-chain construct. This significantly reduced spurious luciferase reconstitution and thus background bioluminescence activity, yielding a much larger signal increase when analyte antibodies were bound to epitope peptides, incorporated into the linker and fused to LgBiT C-terminus. Albumin-containing serum samples exhibited lower background and higher S/N than analyte Abs in buffer alone. However, the level of S/N enhancement has been quite variable, ranging from 2 to 100-fold and yielding overall S/Ns of between 30 and 200-fold, depending on the epitope. If a competing surrogate SmBiT fragment is used instead of a non-specific factor such as albumin, it becomes possible to easily tune strength of the blocking interaction through rational mutagenesis. This design utilizes LgBiT luciferase with SmBiT peptides fused to both termini; the longer semi-rigid linker contains two copies of the analyte Ab binding epitope peptide and is attached to the SmBiT fragment that is also labeled with fluorescent dye (FIG. 9). The ratiometric output of this device comes from a shift in competition between the two SmBiT fragments for their association with LgBiT because extension of this semi-rigid linker, due to bivalent binding of the analyte Ab, disfavors LgBiT association with the attached fluorophore-labeled SmBiT and thus reduces BRET to it from the luciferase. By individually mutating residues in each of the competing SmBiT segments it has been possible to optimize balance between low background and high signal (yielding up to 5-fold S/N), as well as adjust concentration range for analyte response.

This principle of using a large split protein fragment interaction with two alternative, almost identical small fragments, to provide low background and facile tuning of the critical balance between these affinities, is beautifully employed in recent de novo protein devices. The first of these utilized a six-helix bundle with the C-terminal one being interchangeable with a replica helix supplied as a separate polypeptide. Short sequence signals for degradation or nuclear export were embedded within the 6th helix such that they remained sequestered and inactive until displaced by the replica helix polypeptide produced via regulated co-expression to control cellular fate of POI (protein of interest) fused to the switch. In a more recent rendering of this technology, a second split protein was layered on with competing partners. This time the SmBiT fragment of nanoLuc is embedded within the tethered helix (referred to as ‘Latch’) along with a peptide specifying binding to the target analyte. LgBiT meanwhile, is fused to the replica helix polypeptide such that when analyte binding displaces Latch and this peptide moves in, it also pairs to concomitantly exposed SmBiT (in Latch), restoring luciferase activity for a bioluminescent signal of analyte presence. The completely de novo construction of this series of devices enabled these engineers to incorporate clever enhancements such as a preponderance of buried H-bonds at the interface between helix-6 and the remaining helices of the ‘Cage’ fragment so affinity tuning is easy and predictable. Facile construction of biosensors for eight medically relevant target analytes with high sensitivity (limit of detection as low as 15 pM) and up to a 17-fold increase in bioluminescence have been demonstrated. In a similar way, caging of CaM-binding peptide within a tethered low-affinity derivative of the CaM protein, reduced background signal from a dual-chain device that uses simultaneous binding of the chains to target analyte to favor transfer of the binding peptide to an unmutated CaM domain insertion within the output protein module on the partner polypeptide chain.

E) Other Recent Improvements in Split Protein Design

Considerable potential exists for interrogating cellular function of a POI through controlled activation of its split form via assembly driven by chemically induced dimerization of a fused molecular glue system. Unfortunately however, difficulty achieving a low background level of spontaneous split protein reconstitution in absence of the inducer, while also yielding sufficient activation within a reasonable timeframe after adding inducer, prevents its widespread use. To address this short-coming, a two-pronged approach has been developed to maximize chances of generating well-behaved split fragments of a given POI. The SPELL web-based server has been implemented to provide automated prediction of preferred split sites for a given uploaded protein structure. The algorithm is based on comparing summed computationally estimated folding energies of both fragments for splits done throughout the POI sequence, with that of the full-length native protein. Candidate sites filtered for being well-exposed to solvent and in non-conserved regions of the protein, have been ranked to favor lower folding energy shortfalls as well as tighter loops. The second design element utilizes fusion to the FRB/iFKBP system to drive reassembly of the split POI in the presence of rapamycin (or analogs including photocaged for light-activation). The iFKBP truncated version of FKBP12 is destabilizing without rapamycin present, and this lowers propensity of the fused POI fragment to prematurely associate with its complementary fragment and generate interfering background activity in the absence of inducer. Consequently, addition of rapamycin not only favors pairing of split POI fragments by inducing FRB/iFKBP-association, but also promotes folding of the iFKBP-fused fragment due to its stabilization by the bound ligand (and FRB). Another method to computationally optimize split proteins focuses on identifying mutations in its structure that moderately destabilize the molecular interface between complementary fragments, without dramatic impact on overall stability of the reconstituted protein. Since such mutations may inadvertently affect other aspects of POI behavior, this methodology may be better suited to expand output repertoire for synthetic biology than investigating cellular POI function per se.

Emission of visible light by luciferases (when supplied with substrate, ATP, and oxygen) and fluorescent proteins (when illuminated by light of a specific lower wavelength) make them very powerful biosensor output modules for studying cellular biology as well as for analyte detection in vitro. Use of luciferases for research has a long history initially founded mainly on the firefly and Renilla enzymes, but now is more-or-less dominated by nanoLuc, an engineered luciferase derived from deep-sea shrimp and preferred for its relatively small size (19 kD), high stability and roughly 150-fold stronger luminescence. Moreover, split luciferase has really become popular in recent years due to the introduction of the very efficient and robust SmBiT/LgBiT complementary fragment system of the nanoLuc protein. SmBiT and LgBiT correspond to the last strand of the 10-strand b-barrel, and the remainder of nanoLuc, respectively. Recent innovation further splits nanoLuc into a tripartite system of complementary fragments by excising another strand of its b-barrel such that reconstitution now requires association of two peptides corresponding to b-strands #9 and #10, with the remainder of nanoLuc structure (including barrel strands #1-8) provided on a single large fragment. These short b-strand peptides (11 residues each) can be readily synthesized and chemically conjugated to any pair of Abs, or alternatively produced as genetic fusions with minimal impact on expression or stability. And utility of this for facile construction of biosensors based on association of the b9 and b10 peptides, appended to Abs simultaneously bound by analyte, with the large nanoLuc fragment added separately (FIG. 10), has been demonstrated for five different Ab pairs recognizing the EGFR protein analyte. Or in the case of a rapid test for antibodies against SARS-CoV-2, the short b9 and b10 peptides have been added as gene extensions to the termini of the CoV-2 spike protein antigen, and protein G, which bind combining site and constant regions of the analyte COVID Ab, respectfully. Fluorescent proteins, meanwhile, are composed of an 11-strand b-barrel with a central a-helix that contains the auto-catalytically derived chromophore. And like luciferases, they have been extensively engineered to substantially improve handling, most notably to make the superfolder GFP variant. Analogous splitting of this superfolder FP combined with directed evolution to optimize reconstitution efficiency (in three phases) yielded a pair of peptides (about 20 residues each) corresponding to its 10th and 11th b-strands, that could be fused to cellular POIs without negative fallout in terms of protein solubility, stability, or function. Fluorescence intensity upon co-expression of the large (b1-9) FP fragment correlated with independent measures of strength and stability of complex formation for interacting proteins labelled with the b10 and b11 peptides in bacterial and mammalian cells. Moreover, this approach has since been used for many cellular studies of PPI, including screening small molecule pharmacological leads for drug inhibitors of a particular interaction, as well as biosensors for RNA and polypeptide targets based on b10/b11 fusion to non-overlapping binding proteins.

F) Control of Dimerization with Chemical Inducer or Light

The signaling cascades that regulate biological functions of the cell in response to extracellular or intracrine stimuli are often based largely on proximity-driven mechanisms with subcellular location and/or association to other signaling proteins being determinant of downstream pathway activation. Consequently, scientist-controlled manipulation of protein-protein interactions (PPI) and subcellular localization has provided a treasure trove of detailed mechanistic insight into cellular signaling and function. Typically, proximity of POI (protein-of-interest) to a given partner protein, e.g., downstream signaling cascade substrate, is rendered responsive to exogenous input by fusing each to members of a protein pair that are induced to bind one another in the presence of a specific chemical or wavelength of light. In the case of triggered subcellular relocation, such as from cytoplasm to the plasma membrane, induced dimerization is employed to turn on association between partners genetically fused to the POI and a cell anchor signal for the desired recruitment site (FIG. 11). Established protein pairs for chemically induced dimerization, such as the popular FKBP/FRB with rapamycin system, have been utilized via this approach for studies on a wide range of cellular processes as well as therapeutic use for induction of apoptosis to rapidly extinguish CAR T-cells through homodimerization of Cas9 when necessary for patient safety. Light activated systems have unparalleled temporal and spatial precision, with remarkable insights being gained through fusion to light-triggered heterodimerizing protein pairs such as BphP1/QPAS1 [82] or Cry2/CIB1. An alternate approach to create light-activated control of POI proximity to designated cellular protein targets, or subcellular localization, is based on ‘caging’ of signaling peptides within the Ja-helix of the LOV2 protein. This region of LOV2 is generally highly tolerant of amino acid changes and can be engineered to carry sequences specifying cellular localization or binding to a particular cellular protein. Moreover, tight association of Ja-helix to the PAS core of LOV2 in the dark state sequesters this signal, keeping it inactivated until blue light illumination releases Ja-helix from the PAS core, thereby exposing and activating the embedded peptide signal. Finally, light-induced heterodimer formation has also been achieved by analogous LOV2-caging of peptides such as SsrA, a natural binder of E. coli SspB, and the peptide ligands for PDZ or the PDZ-FN3 affinity clamp.

Chemically induced proximity of cellular proteins has also been used to fight disease. In this scenario, labelling the target POI through genetic fusion with an extraneous protein moiety that directs the induced dimerization, may not be ideal. Instead, this therapeutic approach utilizes a chemical crosslinker that has heterobifunctional structure with one half serving as ligand for one protein of the target pair, and the other side for the second. Indeed, this is the approach behind the promising PROTAC (proteolysis targeting chimeric) class of anticancer drugs. These have one end specific for the POI, in this case the cancer mediator protein target for degradation, and the other end is a ligand for E3 ligase; the ternary complex of bifunctional drug bound to target POI on one arm and E3 ligase on the other, leads the later to ubiquitinate the POI, tagging it for proteolytic destruction. Certainly, this chemically induced dimerization for degradation has been a valuable addition to our disease fighting arsenal, however it is important to point out a fundamental distinction between it and the ‘molecular glues’ used above. Unlike the bifunctional reagents which can bind with good affinity to both proteins for proximity targeting, binding of true molecular glues to at least one of the pairing proteins is undetectable in the absence of the other. And secondly, partner proteins for bifunctional reagents do not have any inducer-independent affinity for one another, while those for molecular glues do exhibit low level binding to each other in the absence of the chemical inducer. Coincidently, these are also defining characteristics of the ‘alternate frame folding’ strategy to create de novo ligand-triggered protein switches, albeit using binding partners that are in fact a split protein with competition between alternate complementary fragments. Large split protein fragments in isolation tend to aggregate, and to ameliorate this the Loh group filled the structural gap left by absence of the smaller fragment in their split FN3-HA4 protein (monobody engineered for SH2 specificity) with a ‘stuff’ peptide extension to the larger fragment (FIG. 12). This ‘stuff’ is inferior to the ‘native’ fragment in that it contains mutations that globally destabilize the FN3-HA4 protein as well as one that is central to the binding of the shared ligand but is otherwise normal and still able to yield folded product [90]. The shared ligand, i.e. analyte for this biosensor device, is the SH2 protein domain of c-Abl, and in its presence the stuffer fragment is shed in favor of the ‘native’ small split fragment supplied as a dye-labeled peptide (which has wild type residues necessary to fully restore split protein structure in complex with SH2). The output signal is FRET between a fluorescent dye conjugated to the large FN2-HA4 fragment and that on the small fragment peptide that has paired with the former to reconstitute SH2-binding. Similar devices, except that both the large and small split protein fragments, as well as the competing ‘stuff’ region were all contained on a single polypeptide, have since been constructed for Ca2+ based on its binding to a split EF-hand domain of CaM [91] plus an earlier example using an alternate frame refolding bPBP construct to detect ribose.

Systems for chemically induced dimerization of binding proteins provide a ligand-triggered assembly that can be used as a highly modular input for coupling to a wide variety of outputs. Moreover, this assembly-based mechanism for allosteric communication ensures a substantial conformational input regardless of paucity of natural receptors that exhibit significant structural changes upon binding to the input ligand. Consequently, there has been considerable effort to engineer new ‘molecular glue’ systems that will allow versatile inputs for synthetic biology or creation of biosensors for analytes of commercial, environmental, or medical importance. There are two basic approaches for de novo engineering of ‘molecular glue’ systems: computational and combinatorial, with the starting point of design often a reflection of its end-use. For example, to build a multi-input system for graded response in vivo, one may start with complexes of a viral protease with two clinically approved inhibitor drugs as the prospective chemical inducers because they do not interact with endogenous cellular proteins and have favorable pharmacokinetics. Computer modeling may be utilized to choose a suitable pairing candidate by assessing molecular fit for a range of potential partner proteins (mostly Ab mimetics) to the structure of the protease-drug complexes, followed by initial mutation screening of the partner through the Rosetta suite of macromolecular modeling software and then combinatorial optimization via yeast surface display. Meanwhile, de novo dimerization induced by a toxic intermediate in the biosynthesis of terpenoids has been engineered largely through a computational approach that screened (in silico) some 3500 natural heterodimeric scaffolds for interface structure that is compatible with placement of one of five amino acid clusters predicted to provide good complementarity to the target ligand. Other groups have instead taken an entirely phage display-based combinatorial strategy. Selection is first for nanobody variants captured on immobilized molecular glue target, and then the naïve library is rescreened for variants that bind the first nanobody complexed with the target but are not captured by the first nanobody without inducer of dimerization present. Combinatorial methods may lead to unexpected results, and in the case of a methotrexate-induced dimerization system the second nanobody does not contact this ligand, but instead recognizes conformational changes of the first nanobody that are concomitant with its binding to the methotrexate ligand.

The unique structural features of the ABA-sensing proteins of plants may be utilized to rapidly engineer this natural system of ligand-induced heterodimerization to be specifically responsive to a broad range of alternate chemical inducers. Chemical inducer bound to the ABA-sensing dimer makes the vast majority of its contacts with the PYR1 protein and thus new specificity may be engineered through combinatorial and computation methods focused solely on that partner of the ABA-sensing heterodimer. Computational modelling to remove overly destabilizing mutations, as well as avoiding conserved residues important for receptor activation, guided synthesis of a focused library of some 40,000 double-site mutations within the ligand binding pocket of PYR1. This library has been screened for induced dimerization of the ABA-sensor proteins via their fusion to DNA-binding and trans-activation domains for a readily selectable gene marker, with initial isolates being further optimized through up to four rounds of DNA shuffling and rescreening. Engineering for 38 inducers of two distinct chemotypes, successfully identified PYR1 variants specific for 21, several of which were further demonstrated to function in split luciferase biosensors with low nM sensitivity. However, recognition and binding by the HAB1 (ABA-sensor) partner protein is mediated in part through its crucial interfacial Trp-‘lock’ residue that covers the ligand-occupied pocket of PYR1, making side-chain H-bonds to a conserved water network that is stabilized by a carbonyl group of the inducer ligand and consequently, there is some question as to whether this might limit the range of chemical structures that can act as inducers (regardless of success of these authors for one target ligand without carbonyl functionality and viability of alternate H-bond acceptors in other published work). Meanwhile in another case, fortuitous discovery of homodimeric binding of camelid VHH antibody (i.e. nanobody) to caffeine was made into a very effective molecular glue system through random mutagenesis followed by high throughput in-cell screening to improve affinity [93].

Allosteric Engineering with Protein Switches

In this context, protein switch refers to a distinct change in global structure of a polypeptide in response to the input stimulus. This can take the form of induced interaction between separate domains that are tethered together in a single continuous polypeptide, or a self-contained natural input-responsive element extracted from cellular regulatory or signaling machinery (see sections A and B below, respectively). In contrast to assembly-based designs (discussed above) which can be encoded on one or more polypeptide chains, communication of input module structural switching to the output module(s) requires a covalent connection between them, and consequently these devices are all single-chain. Systems composed of two or more polypeptides must generally contend with a response that is dependent upon absolute and relative concentrations of the components, as well as further complication due to a ‘hook effect’ of lowering response at high analyte concentrations. Recent progress has streamlined the optimization process, but nonetheless two-component assays are considered to be less than ideal for commercialization, at least by some. Finally, allosteric devices in this category are characterized by placement of the protein switch either between or flanking domains of the output machinery, as opposed to being inserted within a protein structural domain of the output module (i.e. domain insertion, vide infra). As a result, their engineering employs either steric blockage of active regions of the output or changes in relative position and orientation of output module domains, imposed by the protein switch.

A) Protein Switching Through Induced Interaction of Tethered Input Modules

In some IF-THEN devices, input-triggered binding interaction between protein domains fused to both ends of the POI create obstruction of the active site or sterically compromise its interactions with downstream effector proteins. For example, affibody (three-helix Ab mimetic) specific for dark state of LOV2 provides light-induced activation of POI's that are flanked by these modules (FIG. 13). This approach has been demonstrated for control of cell shape and motility through light-regulation of the actin-severing cofilin protein as well as aTat, a microtubule acetylating enzyme. Also, disruption of guanine-exchange factor interaction with small GTPase effector substrates, or HCV protease activity, has been achieved via light-triggered dimerization of flanking Dronpa domains, or in the case of the former, also through assembly of flanking PDZ/FN3 affinity clamp domains with its binding peptide. Alternatively, association of interacting input modules embedded within the linker connecting flanking output elements, can be used to restrict orientation of the terminal domains and thereby prevent (or enhance) their productive pairing. For example, phosphorylation of a linker site by cellular signaling kinase, triggers formation of an intramolecular complex with its binding domain, also in the linker, which disrupts productive association of flanking N and C-terminal domains of Firefly luciferase to facilitate direct monitoring of this kinase activity in cell culture and murine tumor grafts. This design paradigm has also been used to release suppression of a tobacco virus protease by an engineered autoinhibitory peptide due to its inability to bind the protease enzyme correctly when assembly of the intervening PDZ/FN3 affinity clamp or FKBP/FRB linker has been triggered by its binding peptide or rapamycin (analog) input, respectively (FIG. 14). Interestingly, alternate spacing between these elements converted the former to being repressed by the PDZ peptide ligand, presumably by inducing an enhanced autoinhibitor positioning in these cases. Also, this approach has been used to prevent pairing of FPs that otherwise weakly heterodimerize for very efficient FRET; designs include pairing FPs linked via the PDZ/FN3 affinity clamp or two small domains that cooperatively chelate Zn2+. A biosensor based on binding of bile acid to its nuclear receptor LBD placed between heterodimerizing FP, uses ensuing input-triggered opening of a binding pocket on the LBD to capture transcriptional co-activator peptide fused to the C-terminus of one FP and disrupt their pairing, leading to a diminished FRET signal. Meanwhile, spectrally overlapping FP that don't dimerize have been fused to both ends of linker carrying CaM and its binding peptide; association of these regions of the linker in the presence of Ca2+ input shortens distance separating the FP and causes an increase in FRET signal. Recent innovations in this design strategy include the creation of a recombinant DNA platform to facilitate linker sequence and length optimization generally necessary to obtain efficient allosteric function.

B) Input Arising from ‘Self-Contained’ Protein Switches

Often, these IF-THEN protein designs utilize input-dependent condensation of a naturally switching receptor module placed in the linker between FP to reduce the distance separating them, yielding an improved FRET signal when bound to analyte. Biosensors for a variety of metal ion analytes have this design paradigm including for Zn2+ by zinc fingers (FIG. 15), rings etc, K+ by E. coli Kbp, Mg2+ by cytoplasmic domain of E. coli CorA Mg2+-transporter, Cu2+ by Amt1 or Ace1, rare earth cations by the CaM-like Ln3+-binding protein lanmodulin or Ca2+ by TnC. Another very popular natural ligand-dependent protein switch is provided by the bPBP class of receptors, which all have the same clam-like structure that closes the upper and lower lobes together when bound to ligand. This family of receptors naturally recognizes a substantial variety of metabolites and FRET-biosensors for maltose, glutamic acid neurotransmitter, glucose, ribose and other sugars/amino acids have been utilized. It's worth noting that several groups have, with varying levels of success, pursued broadening the range of compounds recognized by these receptor proteins through mutagenesis so that the same large ‘Venus-flytrap’ repositioning of lobes might be employed for biosensors of de novo specificity. Several other metabolite-binding proteins that happen to undergo significant structural change upon ligand binding have been incorporated into similar FRET-biosensors for cGMP by CNBD, ATP by ParM, cyclic dinucleotide by STING and ATP by its synthetase from B. subilis. The intracellular loops of GPCR are repositioned in response to agonist binding to this membrane receptor and a FRET-biosensor was constructed by adding donor FP to the third loop and acceptor FP via fusion to the C-terminus. Due to the fairly subtle movements of this protein, change in FRET signal is modest compared to those from bPBPs for example. This FRET-based approach has also been utilized to report activation of cellular Src kinase due to a large concomitant structural change that substantially impacts distance separating FPs fused to its termini. A substantial advantage of these genetically encoded FRET-based fluorescent biosensors is that they can be implemented to dynamically monitor individual levels of these molecules within living cells and organelles with a high degree of temporal and spatial resolution.

Firefly luciferase (FLuc) is composed of two domains with its active site located at the interface between them. Moreover, it produces bioluminescence through a two-step reaction involving a “domain alternation catalytic mechanism” wherein adenylation of substrate in the first step is followed by a roughly 140° rotation of the smaller C-terminal domain to carry out oxidative decarboxylation of this intermediate to produce emitted light. Several biosensors capitalize on this by using various natural protein switches to transduce input stimulus into a bioluminescence response by altering relative placement and flexibility between the N and C domains of Firefly luciferase. For example, retaining natural connection of the domains while fusing FKBP/FRB, the cAMP-binding B domain of protein kinase A regulatory subunit, or a peptide linker that contains recognition site for caspase 3 protease, to the termini of FLuc (on separate domains, yet close together) suppressed bioluminescence in presence of rapamycin, absence of cAMP binding or prior to proteolytic cleavage, respectively. Meanwhile, fusion of isolated N and C-domains of firefly (and Renilla) luciferase to termini of the estrogen receptor LBD led to discrepancies in their relative positioning, and thus differing levels of bioluminescent competency, associated with the distinct conformational changes of the LBD in response to agonist vs. antagonist binding. Another design attached N and C-domains of FLuc to the galactose/glucose-binding bPBP; interestingly, large-scale closure of the binding protein domains to capture ligand was not directly utilized to alter relative positioning of split FLuc fragments, as both were joined to a single lobe of the bPBP protein. Similarly, inhibitor binding to a destabilized cell signaling EED subunit inserted between N and C domains of FLuc resulted in improved bioluminescence apparently from reduced flexibility of the EED-ligand complex preventing excessive movement between the FLuc domains. A related domain mobility-based design approach utilized increased spacing and flexibility of LOV2 domain in response to blue light illumination (see following paragraph) to activate the apoptotic caspase-3 zymogen by mimicking natural processing cleavage in allowing its inherent spring-loaded mechanism to play out for productive realignment of the flanking fragments.

When the light-sensing LOV2 protein is illuminated with blue light its C-terminal Ja-helix dissociates from the rest of the protein and unwinds. This substantially increases the physical separation between the part of LOV2 that has remained folded and the POI N-terminus to which LOV2 was C-terminally fused (can be repositioned by circular permuting POI), and so it should be feasible (FIG. 16) to use this to release steric blockage of the POI active site introduced by a LOV2 fusion that is tight in the dark state. One of the early celebrated demonstrations of this approach fused LOV2 to N-terminus of the Rac1 GTPase. However, this effect is dependent upon spurious weak binding between the LOV2 and Rac1 proteins. Nonetheless, this approach is ultimately successful for the related, but not LOV-binding small GTPase, Cdc42, provided the dark-state stabilized mutant (F56W) of LOV2 is utilized. Moreover, efficacy of this end-to-end fusion of LOV2 to POI N-termini to create steric interference that is released by light, has been demonstrated for caspase-7 to provide rapid induction of apoptosis as well as photoactivated release of mDia formin autoinhibition to promote nuclear actin network assembly. Similar LOV2 fusions have been utilized to regulate Ca2+ flux through the Orai1 channel or transcriptional repression via the DNA-binding REST protein by blocking modulating proteins that stimulate or suppress (respectively) their activities, from interacting with these POIs unless illuminated. Finally, transition from unfolded polypeptide to folded structure analogous to that of the LOV2 Ja-helix but induced by Zn2+ binding to the zinc finger motif, has been used to reduce solvent access to the protein-engulfed chromophore of fluorescent protein that has been circularly permutated with the zinc fingers attached to both new termini. This reduced solvent access to the FP core translates to an Zn2+-dependent increase in fluorescence output signal intensity.

Allosteric Proteins by Domain Insertion

In broad general terms, historic strategies discussed so far generate IF-THEN outcomes by using various means to change proximity or relative orientation of output modules (which may or may not be independently folded protein domains) or create a steric blockage of the active site of the output protein in response to input. The domain insertion approach on the other hand, uses input events to alter the conformation from within a single protein domain output module. Insertion involves fusion of the introduced domain to host protein residues that otherwise would be (nearly) adjacent, and typically it causes some local structural changes and/or destabilization of the host output protein, particularly around the site of insertion. The extent of disruption to the host output module protein fold is then either mitigated or exaggerated by conformational changes to the inserted input module, effectively ‘repairing’ the ‘wound’ site, or in the case of mutually exclusive folding, further compromising output protein structure and activity due to incompatibility of the induced input module structure with that of the host protein. The input ‘domain insert’ can take a variety of forms, from a simple ligand-binding domain or self-contained protein switch, to a tethered pair of protein domains or peptides that are induced to dimerize in response to the input stimulus. And some are not actually insertions, but rather end-to-end fusions that are included here because they involve disruption of structure in the output host protein domain module. Moreover, unlike IF-THEN designs discussed above, where physical changes in relative positioning of the component modules are central to the allosteric mechanism, the domain insertion approach is generally based on more subtle changes of conformation and in some cases appear to largely arise from changes to localized stabilization or entropic motion of the proteins involved, without significant change to their averaged ‘static’ structures.

A) Insertion of the Calmodulin Protein Switch

Calmodulin (CaM) is a small protein (17 kD) that undergoes well-defined structural changes in response to binding Ca2+ and specific peptide ligands, derived from CaM-regulated proteins. In the apo state, CaM has a globular structure with its termini close together (about 9 Å apart), but when bound to Ca2+ it adopts an extended dumbbell-like shape (termini separated by ˜31 Å), that is condensed to bring its polypeptide chain termini back together when the peptide is bound as well. Inserting CaM into a particular surface loop of BLA yielded enzyme that was activated 120-fold by peptide binding to the extended Ca2+-bound CaM structure (FIG. 17). This high signal-to-noise ratio (S/N) comes from a very low background activity for the Ca2+-loaded state of CaM without peptide bound, which is presumably due to the much larger separation of its termini, that are of course fused to nearly adjacent residues of BLA at the insertion site. A similar insertion of CaM into the last surface loop of the 10-strand b-barrel of the nanoLuc enzyme is also too extended to restore native-like positioning of this last b-strand unless CaM-peptide is bound. In this case, the CaM-binding peptide (sequence altered to reduce affinity and background signal) is provided by a separate chain that pairs with the first through simultaneous binding of receptors on both, to the target analyte. Given all this, the converse observation that extended Ca2+-bound state of CaM inserted into a tight hairpin surface loop of GDH is more restorative of the host enzyme activity than the relatively compact apo state of CaM, does seem counterintuitive. However, Ca2+ also plays a direct role in GDH catalysis and impact of its binding to the nearby CaM insert appears to be more nuanced than simple structural disruption of the GDH host by extended separation of CaM termini upon Ca2+-binding. Furthermore, recent studies of Ca2+-bound CaM in solution suggest that its structure is not completely rigid with the central helix that connects the globular domains being bent, at least some of the time. Meanwhile, peptide binding to the Ca2+-loaded state of CaM inserted into a different loop of GDH lead to activation of the output enzyme (to 50% of native vs. essentially no activity without CaM-peptide), consistent with expected amelioration of disruption to surrounding structure via this condensed state of the insert.

Glucose dehydrogenase (GDH) is the modern sensing enzyme of choice for diabetic blood glucose monitors and so integrating this into biosensor design is very attractive from a PoC applications standpoint. Accordingly, the CaM switch triggered by peptide binding to the Ca2+-loaded state has been inserted into rationally selected surface loops of GDH and then evaluated in a two-component biosensor design wherein sandwich assembly of receptors on analyte brings the peptide fused to one, close to GDH-CaM fused to the other, and thus triggers CaM/peptide-complex formation to restore GDH activity. Initially demonstrated using FKBP/FRB dimerization with rapamycin, this was further developed for biosensors of amylase and three other protein targets via their cognate pairs of nanobodies, HSA through protein G and nanobody, as well as methotrexate using nanobodies. Moreover, these authors were able to achieve S/N ratios of almost 200-fold by incorporating Cys residues into the short linkers that connect CaM to GDH. Apparently, this is through spontaneous disulfide formation in the peptide-bound state that locks the compacted state of CaM and provides a ‘ratcheting’ mechanism to increase activation. Furthermore, the Alexandrov group has also developed a modified version of this two component GDH-based biosensor, that uses a low-affinity CaM derivative attached to the peptide-receptor fusion to reduce background by imposing a (modest) thermodynamic barrier to the association of peptide and the CaM inserted into GDH. Obviously, the above designs are dependent upon the substantial conformational switching of CaM, and a similar approach based on considerable ligand-dependent structural change of the estrogen receptor LBD has been utilized to engineer chemogenetic control of the CRISPR-associated Cas9 endonuclease; antagonist binding induces activation, consistent with the much closer positioning of its LBD chain terminal junctions for domain insertion, than in the apo or agonist-bound states.

B) Induced Dimerization of Domains Fused to Termini of the Insertion Site

Binding between input sensor protein elements fused to new output module termini created by its circular permutation, is another approach to biosensor design wherein input-triggered assembly of these elements plays a role analogous to conformational input from the protein switch inserts discussed above. This design paradigm has been used quite extensively to create Ca2+-indicators that capitalize on association of CaM with its target peptide in presence of the metal cation, to modulate fluorescence signal from circularly permuted FP to which CaM and the peptide are terminally fused (for more on FP permutation, see following section). The non-covalent cyclization and activation of output through assembly of input analyte-binding modules fused to the termini of a circularly permuted output protein, has also been utilized for non-fluorescent biosensors. For example, the Alexandrov group created bioluminescent sensors for the rapamycin and tacrolimus molecular glues as well as amylase protein by fusing the respective chemically induced dimerization domains, or non-overlapping nanobodies, to circularly permuted nanoLuc. The permutation strategy mirrored that of Sm/LgBit split of nanoLuc protein, and not only did these designs avoid complications associated with analogous two-component constructs, but the simultaneous analyte-binding by two receptors on a single-chain construct increased sensitivity by 10 to 15-fold. Moreover, they also engineered an important red shift in the output luminescence by utilizing BRET to an attached red FP and therein made the biosensor capable of monitoring analytes in blood-containing samples. Ratiometric biosensors for a variety of protein and small molecular targets were constructed with limit of detection as low as 50 pM and as much as a 16-fold S/N. Furthermore, a parallel design paradigm utilizing terminal fusion of a pair of analyte-binding domains to circularly permuted GDH led to ultrasensitive electrochemical biosensors for small molecules and proteins that can be read using a standard glucose monitor for diabetics. Restructuring at the fusion site has also been achieved by light-triggered assembly of a pair of VVD photoreceptor domains incorporated into a single contiguous polypeptide inserted into a b-hairpin of the Src kinase catalytic domain. Homodimerization of the VVD domains is antiparallel, bringing N and C termini of each quite close to those of its partner, and is thus well-suited to restore proper positioning of normally adjacent host insertion site residues of the b-hairpin. Moreover, molecular dynamics modeling indicated that VVD assembly within this linker concomitantly restored the catalytically important G-loop of Src to native-like structure and dynamics, despite its distance from the insert.

C) Fluorescent Protein as Conformationally Sensitive Output Module

Fluorescent proteins (FP) provide significant opportunity for cellular biosensor development due in part to sensitivity of their optical properties to microenvironment of the normally buried chromophore. The natural termini of FP are relatively flexible and quite distant from the chromophore, and so to yield structural perturbations that are likely to significantly impact fluorescence signal, the natural termini are fused via a long flexible linker, with FP insertion into the input module host protein generally done at sites within the 11-strand b-barrel that encapsulates its chromophore. Designs in this category include insertion of the circularly permuted FP into hinge region of bPBPs specific for glutamate, as well as those engineered for recognition the GABA, serotonin, and acetylcholine neurotransmitters. Meanwhile, insertion of circularly permuted FP into structurally switching regions of the GPCR family of membrane receptors yield biosensors for dopamine, serotonin, norepinephrine and acetylcholine from the cellular neuroreceptors for these agonists. Other similar biosensors have been based on the significant movement of the 4th transmembrane helical segment of the voltage sensitive domain (VSD) of a sea squirt phosphatase. One of these, has a chimera of this VSD and that of the Kv3.1 potassium channel protein inserted into a surface loop of the red FP host, and yielded a modest (3% per 100 mV) increase in fluorescence intensity upon membrane depolarization. Response time of this construct was improved 25-fold by shortening linkers at the insertion site, albeit with reduced signal magnitude and in the opposite direction. Meanwhile, the reverse configuration of circularly permuted FP (cpFP) inserted into the tighter third loop of a VSD homologue from chicken, has led to a voltage biosensor with substantially better kinetics and up to −50% signal change (per 100 mV), following linker optimization and point mutation changes to both protein modules.

Beyond the above biosensors based on global structure changes in the protein switch input modules, high sensitivity of fluorescence signal of the cpFP insert to its chromophore microenvironment also supports several new designs that capitalize on localized structure changes associated with ligand binding to several natural receptor proteins. Often these design efforts start with identification of flexible loops proximal to the binding site that undergo some kind of closure or rigidification concomitant with formation of the ligand-bound complex. For example, a fluorescent biosensor for monitoring relative levels of cellular metabolites ATP and ADP, uses the GInK1 bacterial regulator of ammonia transport with cpFP inserted into its “T-loop”; although the GInK1 protein binds both metabolites, this loop is ordered only in the presence of bound ATP, yielding up to an 8-fold ratiometric (shifted Imax for excitation) response. Similarly for a biosensor of IP4 (a cellular secondary messenger), cpFP insertion into a flexible loop of the pleckstrin homology domain (from Bruton's tyrosine kinase) provided a 1.5-fold change in ratiometric signal. Meanwhile, ligand-triggered movement of the structured “minor-loop” and associated C-terminal b-sheet region of the periplasmic citrate-binding domain of CitA histidine kinase is central to transmembrane signaling by this regulatory bacterial receptor; insertion of cpFP into this loop of the CitA periplasmic domain yielded roughly a 2-fold ratiometric signal for citrate presence. Interestingly, the “switch I” loop of the G-protein domain of the E. coli iron transporter protein FeoB is ordered in the absence of its ligand and becomes disordered upon binding of GTP, leading to a 5-fold ratiometric signal for GTP detection via cpFP insertion therein.

D) End-to-End Fusion of Protein Switch to Output

Conformational switching of the input module can also be used to induce structural changes within the FP output module via a direct end-to-end fusion. This of course, creates a single covalent linkage between the protein modules, whereas the much more common insertional approach entails a pair of crossover points between them, which typically leads to a stronger allosteric coupling. Nonetheless, the high impact of subtle changes to chromophore microenvironment on FP optical properties facilitates utility of this strategy for fluorescent biosensors, albeit with generally modest changes in signal intensity. For example, tandem end-to-end fusions of sea squirt VSD C-terminus to loop or b-strand positions of FP created by its circular permutation, led to quite small dynamic ranges: 1.9 and −1.2%, respectively. However, an analogous fusion of this VSD to red FP, with subsequent random mutagenesis of the entire gene and signal performance screening, did yield an optimized construct exhibiting a 6.6% increase in fluorescence per 100 mV and fast (roughly 3 ms) response kinetics.

The light-sensing LOV2 domain, on the other hand, provides a special opportunity to regulate certain output modules by merging its C-terminal a-helix with the N-terminal helix of an output protein module via an end-to-end fusion of overlapping polypeptides. This design paradigm has been used to engineer a light-regulated trp operon repressor protein in which the shared helix created by fusing C-terminus of the LOV2 Ja-helix directly to a truncated version of the repressors N-terminal helix, is only available to restore DNA-binding function to the latter when illumination favors its dissociation from the LOV2 core (FIG. 18). Further rational optimization to suppress residual DNA-binding activity in the dark by improving stability of Ja-helix docking to the LOV2 core, yielded a roughly 10-fold increase in switching that provides an overall 70-fold increase in DNA affinity upon illumination. A conceptually analogous blue light-activated DNA-binding protein has been built using sophisticated structure-based engineering to create an overlapping end-to-end fusion of the homodimeric bZIP protein, GCN4, to the N-terminus of the blue light-responsive bacterial photoactive yellow protein.

E) Injection of Disorder with LOV2 and Targeting Intrinsically Disordered Regions

The general design paradigm for typical allosteric engineering by domain insertion utilizes inducer-mediated rigidification of the input module to at least partially mend the disruption of surrounding host output protein structure inherent in construction of the insertional fusion. The LOV2 input module, however, has its termini rigidly positioned close (about 10 Å) to one another in the dark state, but when induced by blue light illumination, both the long C-terminal, and short N-terminal, a-helices of LOV2 dissociate from the rest of the protein and become unwound to random coil. Consequently prior to induction, structure of the output protein at the site of insertion approximates that of the native host, being held in place by folded N and C-terminal helical regions of the LOV2 insert, but upon illumination the surrounding host structure becomes destabilized due to disorder ‘injected’ through light-induced unwinding of these helices. Through LOV2 insertion into cellular POI at non-conserved surface loops that were tight, and computationally identified as being allosterically coupled to the active sites, the Hahn group engineered photoinhibited versions of protein kinases, Rho family GTPases and guanine exchange factors (GEFs), as well as activation of Vav2 GEF by blocking function of its autoinhibitory domain. Local disorder ‘injected’ by light-induced unwinding of the LOV2 termini is then propagated to the active site of the output module via the computationally identified networks of allosteric communication. Resultant conformation and extent of activity changes optogenetically induced in the POI of these LOV2 chimeras closely resemble those of the natural proteins, and thus provide spatially and temporally precise modulation of morphodynamic signaling pathways within living cells. Interestingly, similar engineering of an anti-CRISPR protein without benefit of prior knowledge of its 3-D structure identified a particularly effective insertion site that was not only partially buried, but also immediately adjacent to highly conserved residues contacting the Cas9 target of this inhibitor protein; it is thought that while the LOV2 insertion does not sterically block interaction in the dark state, its proximity to this region leads to direct disruption of the binding surface due to destabilization of local structure upon illumination. Meanwhile, LOV2 insertion at 17 positions within the seven solvent-exposed surface loops of a monobody (antibody mimetic based on a type III fibronectin, FN3, scaffold) identified one loop as yielding allosterically active variants at several positions and another loop in which light regulation of binding to the target protein (SH2 domain) was observed for one site when the loop was also shortened by three residues. These loops both connect apposing sheets of the FN3 b-sandwich and illumination of their LOV2 derivatives caused an up to 330-fold reduction in binding affinity, apparently due to altered packing between these sheets which impacts curvature of the outer face to which the SH2 ligand binds. Consistent with the host-destabilizing role played by LOV2 helix unwinding when illuminated, it has been possible to engineer near complete light-induced dissociation of three monobodies from their targets using optimized insertions into loops distal from the binding site, but screening for nanobodies turned ON by light was successful only when two LOV2 domains were placed near the combining site such that they sterically block antigen binding in the dark, but not illuminated, state.

End-to-end tandem fusion to LOV2 can also provide a means to inject disorder into the output protein module for its regulation, in response to light. In protein tyrosine phosphatase (PTP) 1B, an allosteric communication network couples catalytically critical opening/closure mechanics of the “WPD-loop” with conformational state of its a7 C-terminal helix, located some 25 Å away. Closure of the “WPD-loop” on inhibitor bound to the PTP1B active site is matched by a7 transition from a mostly unwound state having fast dynamics, to a stable a-helical structure devoid of significant motion; accordingly, mutational stabilization of the a7 helix increases PTP1B activity, while its complete deletion reduces catalytic function 2.9-fold. Engineering PTP1B for light regulation by direct fusion of the N-terminal A′a-helix of LOV2 to the end of a7 of the phosphatase, led to a 2.5-fold drop in activity (Kcat; Km was not affected) when illuminated due a7-helix destabilization caused by unwinding of the LOV2 N-terminus

Analogous fusion of LOV2 to C-terminus of another member of the PTP family did not lead to its regulation by light unless significant 1B sequence (25 non-contiguous residues) was also incorporated, because its C-terminal helix is not allosterically coupled to “WPD-loop” closure despite 70% identity between the proteins. Insertion of LOV2 into a not naturally switched region of these PTPs, did yield light-regulation levels comparable to the prior end-to-end fusions, albeit with more substantial and unpredictable impact on baseline activities of the insertional fusions in the dark.

Transition of localized regions, or in some cases even entire binding domains, of polypeptide from a highly dynamic, unfolded state that can visit a wide range of fleeting structures called a conformational ensemble, to a single well-defined relatively rigid structure, plays an important mechanistic role in many naturally allosteric systems. Moreover, analogous transition of host protein conformational ensemble resulting from disruptive impact of inserting a ‘loose’ input module(s), to a less dynamic state held more firmly in place by an input module that has become more rigid in response to the input stimulus, is fundamental to allosteric engineering via domain insertion. Nonetheless, domain insertion that specifically targets an intrinsically disordered protein region already allosterically coupled to switching in a biological regulatory system, is not known. In the case of the Kir2 family of inwardly rectifying potassium channels, changes in conformation of polypeptide segments that connect the transmembrane (TM) and large cytoplasmic domains results in a 6 Å movement of the latter toward the pore, with concomitant twisting that splays the inner TM helices apart and opens the channel in response to binding of activator metabolites. Primary of these switch regions is the so-called “tether helix” that is too flexible to be resolved in crystal structures of the OFF, i.e. closed Kir2 channel, but becomes condensed to an a-helix, forming part of the PIP2-inducer binding site, when Kir2 is activated and the channel opened. Insertion of the small (9 kD) cryptochrome-interacting basic-helix-loop-helix domain, Cib81, into this region of the Kir2.1 potassium channel allowed it to be regulated by illumination through its blue light-induced interaction with co-expressed CRY2 protein. In this case, illumination causes already low conductivity of the Cib81-inserted channel to be further suppressed, presumably because Cib81-rigidification upon CRY2-binding causes the “tether helix” conformational ensemble to be shifted away from that supporting Kir2.1 activation. Cib81-insertion at two sites corresponding to regions of conformational switching upon interaction of Kir2-family homolog GIRK, with its G-protein stimulator yield Kir2.1 variants that have been induced to higher levels of conductivity by light.

F) Allostery by Ligand-Induced Stabilization of the Inserted Domain

It is widely appreciated that binding of small molecules or folded polypeptide ligands to receptor proteins generally results in increased stability and reduced flexibility of the latter. This in fact has been utilized to prevent proteolytic degradation of mutated ligand binding modules, and the POI to which they are fused, when complexed with the small molecule protectant. In an effort to engineer allosteric systems that can be readily customized to provide input specificity for potentially any desired activating ligand, randomized libraries of the b-lactamase (BLA) enzyme responsible for ampicillin resistance of recombinant E. coli have been generated and inserted into host monobody and DARPin antibody mimetic proteins, and screened for an ‘antigen’-dependent increase in colony resistance to the antibiotic. In vitro analysis of recovered fusion proteins demonstrated that increased BLA activity was due to stabilization of the antibody mimetic modules through interaction with the MBP ‘antigen’; for the best monobody and DARPin chimeras, there was a 10 and 9-fold increase in activity over that from non-specific control protein added as a mock inducer, respectively. Unfortunately, BLA-insertion substantially compromised binding affinity of these mimetics for their MBP target ‘antigen’ and attempts to create constructs specific for two new ligands for each, by altering binding site residues, was fully successful for only one of the DARPin targets [185]. Meanwhile, truncation of 20 residues from the N-terminus of the FKBP12 chemically-induced dimer partner, converted it to an input modulator of POI activity that is induced by rapamycin, and is suitable for insertion into tight host protein loops because the new termini are only 7 Å apart [69]. This design strategy capitalizes on substantial ordering of these terminal regions when this truncated FKBP insert (iFKBP) is complexed with rapamycin and its partner FRB dimerizing protein receptor (see [69]). And it has been used to generate chemogenetic versions of the FAK [69], PAK1 [193], Fyn, Src, Lyn and Yes [194] protein kinases with induced activities close to that of the wild type enzymes but having more-or-less undetectable levels of substrate phosphorylation without rapamycin present. Moreover, this system for turning ON specific POI with a chemical inducer has been further simplified by fusing its FRB partner protein to the above ‘insertable’ FKBP (iFKBP) such that the former no longer must be provided in trans, and thus avoiding need to balance their expression levels (FIG. 19). Insertion of the resulting ‘uniRapR’ fragment has been used to engineer a variety of rapamycin-induced POI, including catalytic domains of protein kinases, Rho family GTPases, guanine exchange factors (GEFs) as well as binding domain for Vav2 GEF. Finally, behavior of allosteric couplings created through randomized insertion of the cytochrome b562 (Cyt b) heme protein into BLA, provides an important insight regarding general mechanism of allosteric engineering via ligand-stabilization of an inserted input module. In contrast to examples discussed above, switching-competent chimeras recovered from this library all exhibited reduced (>80-fold in one case) BLA catalytic activity when heme was bound by the Cyt b input module. This reversal of coupling polarity is likely a reflection of the disparate conformational character of these inserted modules when in their apo states; molecular dynamics simulations of the iFKBP-FAK allosteric construct indicated that its input module is substantially unstructured, especially in its terminal regions, prior to rapamycin/FRB binding. While for the Cyt b inserted into BLA design, absence of any change in circular dichroism with vs without a 5-fold excess of heme indicates that for the three variants analyzed, allosteric coupling is mediated through a predominantly entropic mechanism, without any substantial change in structure. Evidently, conformational ensemble for the iFKBP-fusions in the apo state are so far-flung from the output structure requisite for catalytic competency that reining it in through tightening of the inserted domain improves output module function, but a more rigid Cyt b structure triggered by heme binding for the other design prevents access to (or between) higher activity conformations within context of a less broad BLA ensemble, that already possesses moderate enzymatic proficiency.

G) Mutually Exclusive Folding

This form of allosteric protein engineering is essentially domain insertion taken to the extreme of complete incompatibility of fully folded insert domain structure with native-like conformation and activity of the host protein, due to a large separation of N and C-terminal fusion sites of the inserted protein domain (FIG. 20). For example, native termini of ubiquitin are about 40 Å apart and consequently insertion of this protein into a permissive 10 Å-loop of the barnase RNase enzyme creates a scenario wherein it is simply impossible for both to simultaneously adopt their correctly folded structures. The differential thermal dependence of folding free energies for these proteins dictates that below about 30° C., the barnase portion is folded while ubiquitin is not, and above this temperature the situation is reversed by barnase unfolding to accommodate the now more stable native structure of ubiquitin (detected by circular dichroism). Moreover, adding a protein inhibitor of barnase (barstar) at elevated temperature (40° C.) causes the otherwise folded ubiquitin module to denature in favor of barnase refolding due to its increased stability through barstar binding. In a related design by the same group, ubiquitin was replaced with the DNA-binding coiled-coil dimeric protein, GCN4. When bound to its AP-1 DNA ligand, ends of the GCN4 helix are separated by 75 Å and so its insertion into the same site of barnase forces the latter to unfold (monitored by Trp fluorescence) and lose RNase function in response to GCN4 DNA-binding. To avoid short-circuiting of this biosensor through spontaneous intermolecular pairing that allows both modules to adopt native-like structures (FIG. 20), it has been necessary to include a low concentration of denaturant in the assays. A light-regulated affibody (Z-domain based antibody mimetic; [45, 103]) has been created by inserting the light-responsive PYP protein domain into the loop connecting 2nd and 3rd affibody helices. Natural termini of PYP in the dark are separated by about 3-times the distance between junction site residues in the affibody host protein, and this diminishes affinity for its target by about 100-fold; illumination causes PYP to adopt a loose molten-globule conformation that relaxes structural disruption somewhat, yielding a 2 to 6-fold increase in target binding strength (vs dark). Short-circuiting of the switch was prevented through intermolecular pairing by using a low concentration of the switch protein combined with mutagenesis and truncation of the 3rd affibody helix.

H) Engineering Workflow

In the case of domain insertion, there is the immediate question of what host protein acceptor sites should be chosen to maximize potential for success. Often, especially in early work, this question has been addressed in a completely empirical fashion, by inserting at random host protein sites (sometimes combined with variable circular permutation of the insert) and then screening the resultant ‘library’ for variants having the desired allosteric function. This entails screening very large libraries and thus requires high throughput methods by which to quickly assess allosteric function. Often screening is based on conditional host survival. Bacterial host survival has similarly been used to screen for allosteric modulation of BLA activity engineered through randomized insertion of cytochrome b562 and DARPin/monobody antibody mimetics. Meanwhile, cancer-biomarker activated prodrug-converting enzymes thymidine kinase and cytosine deaminase were recovered from a library of random insertions of the biomarker-binding domain (CH1 from human p300 protein, which binds hypoxia inducible factor 1a) using a two-tiered host survival screening. The first screen removed those variants with high prodrug-converting activity in absence of the cancer-biomarker through product cytotoxicity from an added precursor substrate, and then in the second screen, allosterically active variants were recovered through complementation of a host-deficiency in these essential enzymes, in presence of the biomarker input.

Fluorescence-activated cell sorting (FACS) by flow cytometry is a selection methodology that permits en masse parsing of libraries into pools of active/inactive variants that can then be rapidly assessed for enrichment of even low abundance clones by next-generation (NGS) DNA sequencing. The approach has been used to recover variants that were successfully folded and expressed in the presence of the maltose inducer, from a random library of cpFP-insertions into the maltose-binding bPBP; these variants were then further screened directly for allosteric function by assessing inducer-mediated changes of fluorescence signal in 96-well plates. Permissive sites of the CRISPR-associated Cas9 protein were similarly identified using FACS to recover random PDZ-insertion variants that retained gene editing function of the Cas9 module, assessed through their ability to repress red FP production by editing its gene from transformed cells. Subsequent extension of this to capture Cas9 variants activated by antagonist ligand input, from a library of randomly inserted estrogen receptor LBD, recovered one which also happened to coincide with a permissive hotspot identified in the prior PDZ insertional screen. Meanwhile, by using fluorescently labeled antibody to tag cells displaying FLAG-epitope incorporated into an extracellular loop of the Kir2.1 potassium channel, it was possible to rapidly assess for correct folding, assembly, and trafficking of this host protein with randomized insertions throughout, via FACS/NGS. Parallel screenings with three model insertion domains having a range of physiochemical properties may provide a measure of ‘differential permissibility’, with some sites accepting all, some accepting none and other sites being permissive for a subset of the inserted domains. The latter group may map to protein regions that are “poised” to undergo order/disorder transitions central to allosteric behavior, and a strong correlation between these differentially permissive sites, and structural features involved in allosteric regulation of the Kir2.1 potassium channel and its homologs has been observed.

The combinatorial methods discussed above do sometimes yield surprising results, identifying allosteric insertion sites that exhibit poor consistency between even quite homologous proteins and would have been difficult to predict otherwise. However, the combinatorial methods are also quite labor-intensive and are contingent upon methodology for very high throughput screening. Consequently, considerable effort has been focused on developing computational approaches to identify insertion sites that have good allosteric potential. Early attempts to do this utilized statistical coupling analysis of numerous protein family sequences to uncover networks of residues that co-evolve with the active site and subsequently, demonstrated its utility as a tool for allosteric engineering. Alternatively, sites of high allosteric potential have also been chosen based on molecular dynamics simulations to highlight groups of residues that move in concert with one another and the active site. These methods are somewhat complicated and recently a more user-friendly approach reliant solely on structure of the host protein for insertion, has been made freely available through an online portal. This is an allosteric network modelling algorithm that converts residue contact matrix extracted from the 3-D structure to a perturbation propagation matrix that is used to model repeated perturbation of the active site to identify allosteric hotspots on the protein surface. An important counterpoint to these computational approaches, especially considering possible disruptive impact of the domain insertion event itself, arises from ongoing debate regarding the plastic and redundant nature of the allosteric network pathways upon which they are based, as well as the degree to which central residues on these pathways are highly conserved by evolution.

Regardless of how the initial site for domain insertion is chosen, subsequent optimization of preliminary candidate constructs is generally key to obtaining an acceptable level of allosteric performance. Even subtle changes in sequence at the junction sites and in fact much further away as well, can dramatically impact coupling behavior. A light-responsive potassium channel has been engineered using combinatorial methods based on K+-transport deficient yeast. A first round of selection yielded a single variant configuration of the LOV2 and Kcv channel polypeptides that exhibited light-inhibition of potassium ion conductivity, but only after fusing it to a peptide that becomes myristoylated in vivo and thus anchors the LOV2 module in lipid bilayer adjacent to the channel. This already is a fine demonstration of a structurally logical switch-to-switch mechanism wherein formation of the LOV2 Ja-helix in the dark pulls (if anchored) the “slide helix” of Kcv and opens the channel, while illumination relaxes this pressure, allowing the channel to close. However, the objective was a K+-channel that opened under blue-light illumination, and so this chimera was randomly point-mutated and screened for that polarity of coupling using their colony/no colony high throughput methodology. And through a single round of selection, light-OFF polarity of the initial construct was converted to a channel activated by illumination, with over twice the magnitude of response (this was a double-mutant variant that included neutralization of the myristoylation signal peptide). Allosteric function was further improved, in particular dark state leakiness, by truncating Ja at the fusion site, and demonstrated light-induced behavioral changes in live zebra fish larvae expressing this channel construct.

‘Top-Down’ Allosteric Engineering

Strategies of allosteric engineering discussed so far are ‘bottom-up’ in the sense that they use input modules, such as analyte-binding or light-sensitive receptors, to control relative positioning, substrate access or local structure of the output module(s). Often their mechanism is not directly related to any natural means by which the output protein module might be regulated. ‘Top-down’ allosteric protein engineering on the other hand, starts with a naturally allosteric system and aims to either alter specificity of the regulator binding site to match a desired input, or create a design wherein the input stimulus alters balance between alternate, pre-existing ON and OFF structural states of the output module.

A) Altered Regulator Specificity

Efforts to modify regulator specificity of naturally allosteric systems will generally either leverage off diversity found within large protein families, or mutagenesis guided by combinatorial and/or computational methods. Allosteric transcription factors for example, have a modular structure with DNA and regulator-binding functions carried out by separate domains, and several research groups have engineered alternate regulator specificity for control of a given gene promoter by simply swapping domains with other members of this large family of proteins. For de novo regulation through mutagenesis on the other hand, the allosteric transcription factor is typically configured to drive expression of a fluorescent protein, and FACS-facilitated high throughput screening is then used to alter its regulator specificity via combinatorial engineering. Of course, mutagenesis necessary to alter specificity must not simultaneously knock out allosteric coupling to the DNA binding domain, and so success is maximized by selecting a starting family member with regulator specificity close to that being targeted. Similar swapping of regulator binding functionalities between members of related proteins, as well as combinatorial mutagenesis, has been applied to nicotinic and muscarinic neuroreceptors as well as the phytochrome and PAS families of proteins. Nonetheless exchanging regulator binding and effector modules between homologous proteins is not always straightforward, but recent insights gained through advanced computer modeling as well as direct experimentation, has substantially improved allosteric network analysis and is leading to higher fidelity in these attempts to engineer new mergers of allosteric pathways.

Creation of new allosteric transcription factors for gene regulation has been extremely useful for the metabolic engineering of organisms for production of value-added compounds, as they provide basis of intracellular biosensor circuitry for dynamic control of the biosynthetic pathway. Furthermore, these engineered transcription factors can be used to provide a selectable phenotype such as fluorescence, antibiotic resistance, or auxotrophic complementation for laboratory evolution of improved production strains or enzymes, via expression of the corresponding gene induced by the metabolic product or intermediate of interest. Meanwhile, domain swapping within the PAS protein family enabled light-control of gene expression in modified cell-free extracts of E. coli, through regulation of PAS histidine kinase activation of the cognate transcription factor. Additionally, biosensors useful for the detection of a variety of small molecules have been constructed using allosteric regulation of transcription factor DNA-binding to directly provide fluorescence output signal or indirectly via cell-free production of a short RNA transcript that interferes with dye-binding, and thus fluorescence, of aptamer oligonucleotide added to the assay. Finally engineering neuroreceptors for activation by orthogonal ligands has been instrumental in the unambiguous assignment of receptor function as well as in development of powerful neurological therapeutics.

B) Modulation of Natural Equilibrium Between ON and OFF States

The classical Monad-Wyman-Changeux (MWC) model characterizes allosteric phenomena as being caused by a shift in equilibrium balance between alternate structural states of a protein, that differ in catalytic competency and is facilitated by preferential stabilization of one state due to its interaction with the allosteric input (eg regulatory ligand binding). This is predominantly an ‘order-to-order’ transition, with structures of the alternate states being mostly well-defined and distinct, albeit often with concomitant changes in protein dynamics as captured by the R (relaxed) and T (tense) state designations originally used in MWC. Recent discovery of the important role played by intrinsically disordered protein regions in many cases of natural allostery however, has led to the Ensemble Allostery Model (EAM), which emphasizes disorder-to-order transitions in allosteric behavior. Bottom-up approaches to engineered allostery, particularly domain insertion, are generally largely dependent on EAM mechanisms. Yet MWC remains relevant, with model updates accommodating subsequent experimental observations such as existence of more than two well-defined alternate structures for some allosteric proteins, as well as possible involvement of metastable intermediates in the transitions between them. Moreover, long-standing capability to raise antibodies that exclusively recognize one or the other alternate conformational states of a wide variety of allosteric proteins, speaks to the mostly ordered and distinct structures for both. Recent examples of this include monoclonal antibodies that bind to ON or OFF states, and modulate their relative stabilities, for an orphan tyrosine kinase receptor, SARS-CoV-2 spike glycoprotein, and a bacterial adhesin protein. Despite this, efforts to engineer allostery from a top-down perspective, by creating ways for novel inputs to modulate the equilibrium balance between alternate pre-existing states of a naturally allosteric output protein module, has been quite limited.

In an approach conceptionally aligned with shifted energy balance between ON and OFF conformers resulting from state-specific binding of natural regulatory ligands (vide supra), a few groups have engineered allosteric systems responsive to non-native inputs through their impact on relative stabilities of the pre-existing ON and OFF conformational states. Reported examples of this can be grouped into three mechanistic categories: i) utilizing input-induced changes in properties of a chemical conjugate ii) using molecular ‘stitches’ to restrict motion and iii) a ‘switch-to-switch’ configuration. MscL is a homopentameric bacterial membrane channel that is tightly closed under resting conditions, but when subjected to membrane stretch associated with cell exposure to hypoosmotic fluids, spontaneously adopts a very wide pore conformation that protects the host from lysis by allowing cytosolic contents, including even small proteins, to escape. By covalently attaching a chemical conjugate that becomes charged upon pH-shift, or light-exposure, to a protomer residue lining the channel lumen, the Feringa group was able to use these inputs to induce MscL opening because the substantially wider separation of the conjugated residues in this conformer, relaxes charge-charge repulsion imposed on the closed state (FIG. 21). Molecular ‘stitches’ refer to engineered input-induced (or released) crosslinks between protein regions that normally undergo significant changes in separation concomitant with the natural ON-to-OFF structural transition. Both ion-channel type and G-protein-coupled glutamate neuroreceptors undergo a significant closure of the agonist binding pocket that is linked to their activation upon formation of complex with this ligand, and it was possible to artificially induce this pocket closure, and receptor activation, by coordination of added heavy metal cation (input) with histidine residues introduced into lips of the binding pocket. Similarly, electrochemical reduction (input) breaks disulfide ‘stitches’ positioned to trap an bPBP-based maltose sensor module in its natural OPEN or CLOSED states, and thereby provides novel means for regulating activity of the BLA output in an allosterically coupled bPBP-BLA chimera combinatorially engineered previously. Finally, physical coupling of an input module that exhibits conformational switching to a naturally allosteric output such that changes to its structural state induce a corresponding shift toward a particular conformer of the latter, is a ‘switch-to-switch’ approach. For example, agonist-induced movement of the C-terminal region of two GPCR-type receptors was used to control opening of the Kir6.2 potassium channel via fusion to its N-terminal “slide helix.” Meanwhile hybridization-induced stiffening of single-stranded DNA fragment conjugated to the output protein module at both its 5′ and 3′ ends, yielded complementary oligonucleotide (input) regulation of guanylate kinase by controlling natural closure of its active site around the GMP substrate, as well as conformational changes normally associated with cAMP binding to the regulatory subunit of Protein Kinase A. An important caveat to these designs is the possibility of evoking a localized unfolding/destabilization mechanism (discussed above under “Allosteric Proteins by Domain Insertion”), which can alter polarity or kinetic parameters of the response.

Antibody: The Prospect of Fully Customizable Input Specificity

Not only can an antibody of basically any desired molecular specificity be prepared with relative ease via animal immunization or recombinant means, but also a plethora having medically important specificities have been prepared already and are thus available for engineering by simply using the cognate antibody protein sequence as raw material of construction. For this reason, antibodies really do make a very attractive sensor, i.e. input receptor module, with which to construct novel allosteric devices. Yet few protein engineers have used antibodies (or their recombinant derivatives: scFv and nanobody) in this role (vide infra); much more common is the reverse, wherein the antibody binding activity is controlled by an operator-provided input. Furthermore, many of these constructs modulate antibody activity through direct occlusion of the antigen-binding site (i.e. combining site) or modifications to the CDR polypeptide loops that form it. To facilitate control of the inhibition, fusion of an obstructing domain or peptide to the antibody, either directly or through a bacterial anti-immunity protein (vide infra), is done via a linker that can be cleaved by input, generally matrix metalloproteases (associated with disease state), but also light. Fusion of an obstructing domain to the N-terminus of VH (is close to the combining site) was moderately (50% inhibition) effective when the latency-associated peptide of TGF-b was used, but much less so for two other pro-domains tested; the first design was then successfully transferred to a second antibody, which also had specificity for a large protein antigen. Alternatively, the blocking entity can be a peptide that competes for the antigen-binding site either selected recombinantly using combinatorial display methodology or directly derived from the antigen epitope. For the latter, fusion was not directly to the antibody itself, but to Protein L, an anti-immunity protein that recognizes and binds to the antibody framework. When the Protein L-to-antibody binding was also ‘locked’ by covalent crosslinking, this construct exhibited a 9-fold drop in affinity that was completely reversed by the protease input, via its cleavage between the epitope peptide and Protein L. Finally, Protein M is an anti-immunity protein recently discovered in a STD bacterium that widely blocks antigen-binding to many IgG subtypes by placing its C-terminal domain ‘over’ the combining site, while being held in position through direct binding of the N-terminal domain to nearby non-variable surface of the antibody. Protein M has been converted to protease-activated antibody-ON switch by inserting appropriate cleavage recognition sequence between the framework-binding (N) and antigen-blocking (C) domains.

Meanwhile, Ab-switching modifications to the CDRs include a variety of means to make the local structure or biochemical properties of these protein loops change in response to inputs such as metal ions, pH, light or phosphatase. For example, photocaged Tyr residues introduced into the antigen-binding surface of four nanobodies by using expanded genetic code methodology, resulting in a 103-104-fold reduction in affinity (measured using an in vitro assay) that was completely reversed upon exposure to UV light. However, a somewhat lower level of inhibition (20-fold) has been obtained for each of two interfacial Tyr residues of an EGFR-binding nanobody, using the same photocage adduct, also measured in vitro. Interestingly, efficient photocage inhibition of antigen-binding within living C. elegans worms required additional combining site mutations, despite good efficiency for apparently the same starting construct in vitro as well as within mammalian cells in culture. Alternatively, metal-ion dependency of antibody activity due to its coordination by residues of the third heavy chain CDR was achieved using Ca2+-dependent capture from a naïve phage display library as well as sophisticated rational design followed by yeast display, for Zn2+. Regardless of metal-triggered increases in affinity of an order of magnitude or more, these designs are nonetheless plagued by quite low binding activities even when induced. Modifications that cause antibodies to lose binding activity when exposed to the relatively low pH environment of lysosomes are of interest to foster their recycling during immunotherapy, and these have been engineered by combinatorially introducing His residues into the CDRs. Meanwhile phosphate group covalently attached to a CDR Cys mutant of anti-lysozyme nanobody, imparted phosphatase-dependent activation of its binding function. Finally, domain insertion of circularly permuted DHFR into the third CDR of anti-GFP nanobody was minimally disruptive in the absence of DHFR ligands, but yielded undetectable GFP-binding due to steric interference from the much more rigid structure of DHFR when stabilized by bound cofactor and inhibitor; however attempts to use this design for three other nanobodies either prevented folding or severely impacted basal activity, but was successful for a fourth (70-fold induction).

Nanobodies are single-domain immunity proteins derived from the Ab of camelids and are structurally homologous with the heavy chain variable domain (VH) that is normally paired with VL in scFv and conventional IgG antibodies. In addition to the combining site modifications discussed above, nanobodies have also been engineered for light-regulation via insertion of the LOV2 domain into surface loops on the non-CDR end of its b-sandwich. Consistent with observations of other LOV2 domain insertions, these yielded an up to 4-fold drop in affinity due substantially increased flexibility of LOV2 regions proximal to the junctions, upon illumination with blue light. However, LOV2 insertions into a non-CDR nanobody loop that is nonetheless close to the combining site led to a light-ON polarity with up to a 7-fold enhancement in affinity, or when combined with a second LOV2 fusion to the N-terminus for a different nanobody (also close to the combining site), an 80% increase in ratio of captured antigen vs. free species when illuminated, in live cell assays. Meanwhile, splitting an anti-GFP nanobody at a surface loop on the non-CDR end of the molecule and fusing the complementary protein fragments to the FKBP/FRB-dimer, yields a 1,000-fold reduction in apparent affinity that is fully restored by rapamycin. The same design approach was then successfully adapted to engineer photo-induced systems, albeit with slow response kinetics (requiring up to an hour to saturate) and without reversibility, for all six nanobodies tested using the Magnet light responsive heterodimer. Moreover, this approach also worked for the one scFv tested, but only if the split was within VH as the analogous construct split within VL was inactive regardless of illumination.

Unlike nanobodies, the antigen-binding site of scFv (and IgG) is composed of protein surface from both the VH and VL domains, which are normally intimately paired to form the Fv, or variable region, ‘superdomain.’ And several groups have capitalized on this to achieve input-regulated scFv binding activity by controlling the VL-to-VH pairing. In silico molecular modelling has been used to design mutation of three conserved bulky residues at the V-domain interface (to Gly, and Ala in one case) as well as find a druglike chemical ligand that fills the resulting cavity. In absence of the cavity-filling ligand, this mutant scFv adopts an ‘open’ configuration with the V-domains no longer interacting directly, and consequently its binding activity was substantially diminished (EC50 increased 10-fold); the cavity-filling ligand meanwhile acts as a ‘molecular glue’ and when added at 0.1 mM, it partially restored activity of this scFv (EC50 dropped 3-fold, but 10-fold for a second scFv tested). The V-domains (VH and VL) of scFv are connected by a long flexible linker, typically composed of -Gly4Ser- repeats, and some scientists have replaced this with switch polypeptides that can be used for regulation by controlling proximity and/or relative orientation of the V-domains. Included here is an anti-lysozyme scFv that exhibits a five-fold increase in affinity in presence of Ca2+ due to improved proximity of the V-domains because of induced assembly of the CaM/M13-peptide complex from these modules contained within the V-domain linker. Similarly, replacing the V-domain linker of six tested scFvs with circularly permuted CaM (without the binding peptide being included in the linker) yielded significant CaM-ligand peptide mediated induction of binding activity for five (up to a 4-fold increase in affinity) in the presence of Ca2+. Finally, is the finding that replacing the V-domain linker of anti-fluorescein scFv with elastin-like peptides led to modest (up to 50%) differences in its affinity response to heat and salt when compared to the same scFv with length-matched standard Gly4Ser-type linkers. Mechanistic interpretation however, was complicated by preponderance of domain-swapped dimers (i.e. diabodies) in the scFv preps, as well as opposite impact of these inputs despite both conditions inducing a similar random-coil to b-turn conformational switch in isolated elastin-like peptides.

All known designs exploiting the inherent versatility of antibodies as a standalone (i.e. not combined with a second receptor for assembly, or using displacement of a surrogate ligand) input receptor for allosteric devices, have used essentially the same approach. It capitalizes on the shared role that both VH and VL domains play in binding the target to make the antigen/hapten behave as a ‘molecular glue,’ driving association of separated V-domains in the presence of the analyte (i.e. antigen/hapten). Early attempts utilized reconstitution of split b-galactosidase enzyme fragments fused separately to V-domains of Abs (FIG. 22) specific for a small molecule hapten and lysozyme protein. Subsequently this design was converted to a single-chain format with V-domains of two tested antibodies being fused to new termini created within a catalytically important surface loop of b-lactamase (BLA), by circular permutation. The engineering itself did severely compromise b-lactamase enzymatic function, but nonetheless modest (up to 2-fold) activation in the presence of peptide and small molecule ligands of these antibodies was observed. Subsequently, efficiency of the former osteocalcin peptide binding design was improved to yield an almost 5-fold induction by first circularly permutating the V-domains such that their junction points with the output module (b-lactamase) are close together in the reconstituted Fv superdomain structure, as opposed to the substantial separation of natural V-domain termini, used for the previous fusions. This has also elevated propensity of the V-domains to spontaneously pair in the absence of the inducing antigen, and it was necessary to include denaturants to lower baseline b-lactamase activity. The same anti-peptide antibody was then the basis of a sensitive and user-friendly protein complementation assay that entails nanoLuc reconstitution from regions on three separate polypeptides. One is simply the entire nanoLuc sequence minus the last two b-strands (11 residues each) of its eleven-strand barrel structure, and the other two are VH and VL fused individually to each of the missing b-strands of nanoLuc. Bioluminescent signal was close to background without analyte peptide present and increased proportionately with the added antigen, ultimately going as high as 88% of that of fully intact nanoLuc protein and easily providing detection of 50-5000 nM antigen peptide by naked eye [295]. Meanwhile, the fragmented output module (i.e. split enzymes) used with two of the above antibodies was replaced with VH/VL-pairing induced reconstitution of b-glucuronidase (mutated at interface residues) through dimerization of subunit dimers to assemble the active homotetramer. Subsequently, a two-fold improvement in sensitivity of one of these was achieved by using a thermostable version of b-glucuronidase with additional mutations at the dimer-dimer interface. And further enhancement in thermostability and performance was then arrived at through combinatorial methods, albeit only tested with the dimeric binding of nanobody to caffeine. Thus far, the above VH/VL-pairing approach to engineering allosteric input has been demonstrated for a small repertoire of mainly peptide and hapten-binding antibodies that were chosen for their efficacy in open-face ELISAs (also dependent upon this antigen-induced association of V-domains).

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to s system for responding to the presence of an antigen or hapten analyte. The system may include an allosteric multi-subunit protein and an antibody. The allosteric multi-subunit protein may have a first conformational state associated with a first biological activity and a second conformational state associated with a second biological activity. The antibody may include a heavy chain variable domain subunit and a light chain variable domain subunit. The heavy chain variable domain subunit of the allosteric protein may be fused to a first subunit of the allosteric multi-subunit protein. The light chain variable domain subunit of the allosteric protein may be fused to a second subunit of the allosteric multi-subunit protein. The first biological activity may be observable when the antigen or hapten analyte is bound to both the heavy chain variable domain subunit and the light chain variable domain subunit. The second biological activity may be observable when the antigen or hapten analyte is not bound to either the heavy chain variable domain subunit or the light chain variable domain subunit.

The allosteric multi-subunit protein may be fused to the heavy chain variable domain subunit with a linker polypeptide. The allosteric multi-subunit protein may be fused to the light chain variable domain subunit with a linker polypeptide.

The heavy chain variable domain subunit may be fused to a first amino-terminal end of the allosteric multi-subunit protein and the light chain variable domain subunit may be fused to a second amino-terminal end of the allosteric multi-subunit protein.

The heavy chain variable domain subunit may be fused head to tail with a first naturally occurring end terminus of the allosteric multi-subunit protein.

The light chain variable domain subunit may be fused head to tail with a second naturally occurring end terminus of the allosteric multi-subunit protein.

In one embodiment, at least a portion of a sequence of the allosteric multi-subunit protein may be removed.

The heavy chain variable domain subunit may be fused head to tail with a new end terminus of the allosteric multi-subunit protein created by a rearrangement of a gene coding region.

The light chain variable domain subunit may be fused head to tail with a new end terminus of the allosteric multi-subunit protein created by a rearrangement of a gene coding region.

The first biological activity may be an enzyme activity having a first level and the second biological activity may be the enzyme activity having a second level different than the first level.

The first biological activity may be a membrane channel opening having a first permeability and the second biological activity may be the membrane channel opening having a second permeability different than the first permeability.

The membrane channel may include a domain residing external to a membrane wherein the domain affects the permeability of the membrane channel.

The allosteric multi-subunit protein may include MthK. MthK may further include cytoplasmic gating-ring octamer.

The first biological activity and the second biological activity may be observable by detecting a change in permeability by measuring electrical conductivity caused by a passage of a plurality of potassium ions through the membrane channel.

The membrane channel may not include a domain external to a membrane.

The first biological activity protein binding affinity may have a first level and the second biological activity may be the protein binding affinity having a second level different than the first level.

### BRIEF DESCRIPTION OF THE DRAWINGS

Some embodiments of the present invention are illustrated as an example and are not limited by the figures of the accompanying drawings, in which like references may indicate similar elements.

FIG. 1 is a diagram of a system for responding to the presence of an antigen or hapten analyte according to an embodiment of the present invention.

FIG. 2 is a diagram of the system of FIG. 1 in combination with a membrane.

FIG. 3 depicts an analyte sandwich with Ab or receptors reconstitutes attached split enzyme.

FIG. 4 depicts assembly of split enzyme on bivalent antibody via attached epitope peptides.

FIG. 5 depicts interaction between cellular proteins I and II inducing FRET output signal.

FIG. 6 depicts that antibody binding to duplicated epitope peptide straightens the linker and activates enzyme output by separating it from the inhibitor.

FIG. 7 depicts that monovalent binding constrains epitope-containing linker and prevents inhibitor interaction.

FIG. 8 depicts surrogate ligand displaced from Ab by analyte triggering a variety of outputs.

FIG. 9 depicts that Ab-induced linker straightening favors LgBiT pairing to SmBiT-2 and reduces luciferase BRET to dye (pink star) conjugated to SMBiT-1.

FIG. 10 depicts simultaneous binding to analyte inducing reconstitution of luciferase output.

FIG. 11 depicts chemically induced dimerization used to control subcellular location of POI.

FIG. 12 depicts input analyte recruiting binding-competent fragment (unshaded), thus enabling FRET between the dye conjugates (stars).

FIG. 13 depicts light releasing steric blockage of the active site by disrupting LOV2 interaction with affibody specific for its dark-state structure

FIG. 14 depicts peptide-induced FN3/PDZ binding disrupting enzyme/inhibitor complex.

FIG. 15 depicts Zn-finger folding to bind the metal condensing the linker, thereby increasing FRET output.

FIG. 16 depicts light-induced unraveling of the Jalpha-helix releasing steric blockage to substrate access.

FIG. 17 depicts conformation changes of CaM insert and its impact on activity of host output protein.

FIG. 18 depicts illumination shifts of shared alpha-helix from LOV2 to trp repressor.

FIG. 19 depicts inducer binding orders insert, restoring activity to host protein.

FIG. 20 depicts ligand stabilization of insert forces either unfolding of the host or its intermolecular pairing.

FIG. 21 is a graph of conjugate ionization by input selectively destabilizing the closed state of MscL.

FIG. 22 depicts an antigen induced pairing of Ab V-domains reconstitutes split enzyme.

FIG. 23 depicts an actameric MthK-Fv channel protein.

FIG. 24 depicts a sequence of non-coding DNA.

FIG. 25A depicts binding curves.

FIG. 25B provides a summary of data.

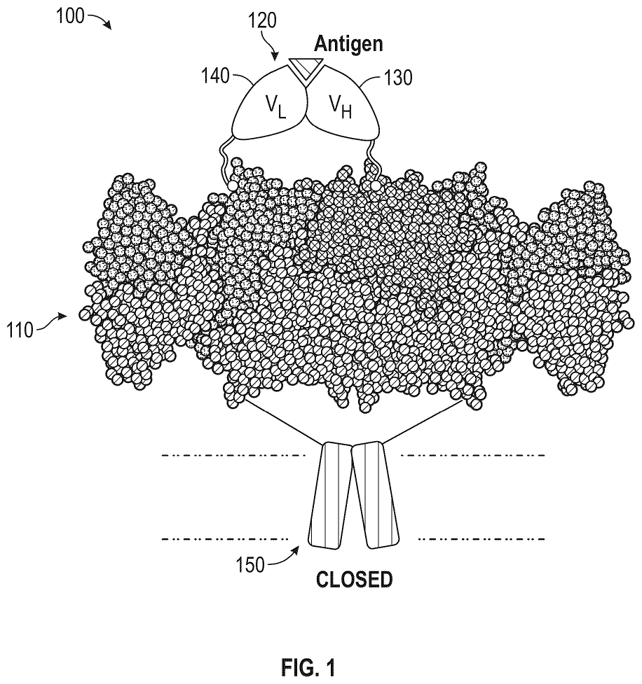

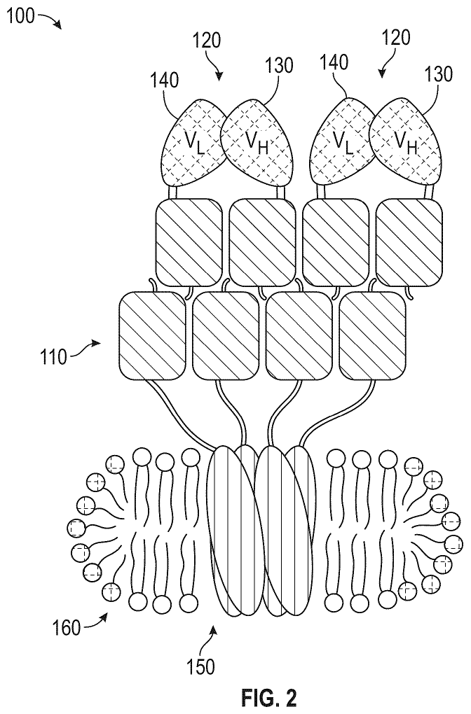

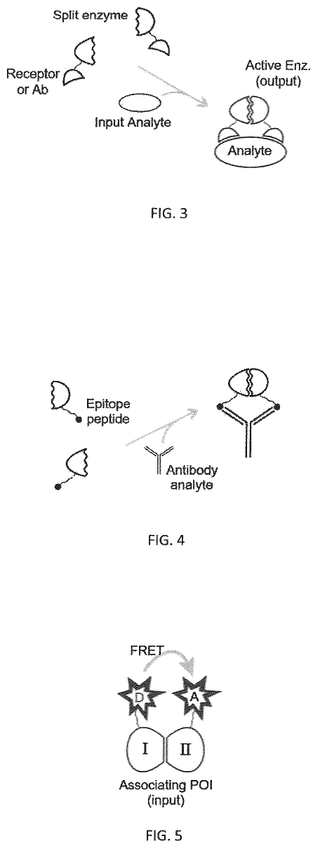

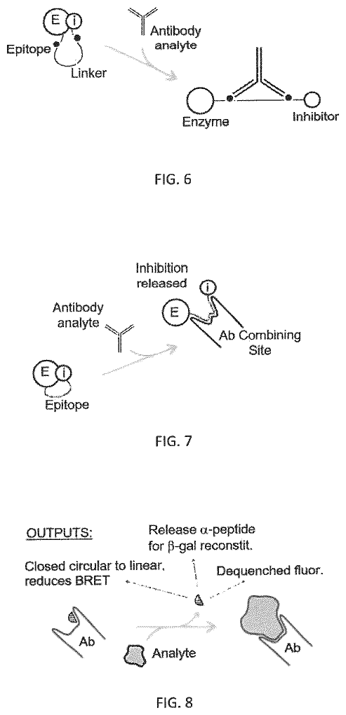

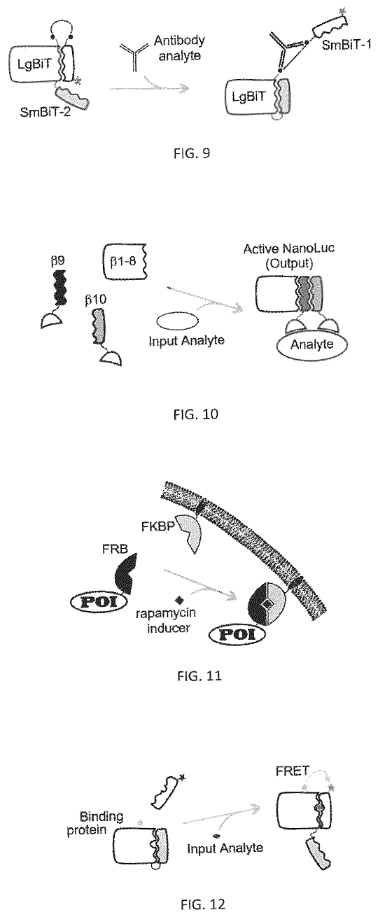

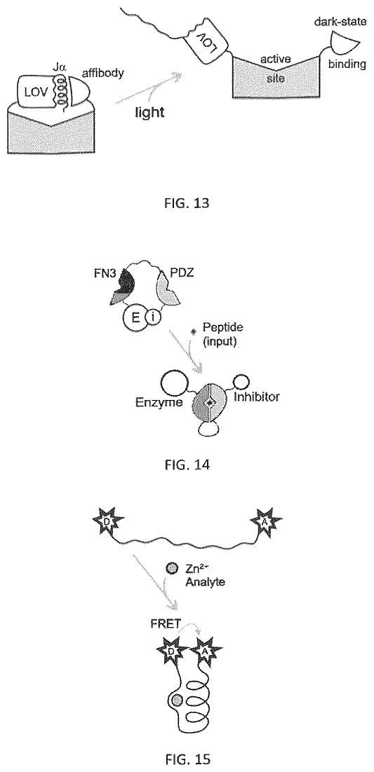

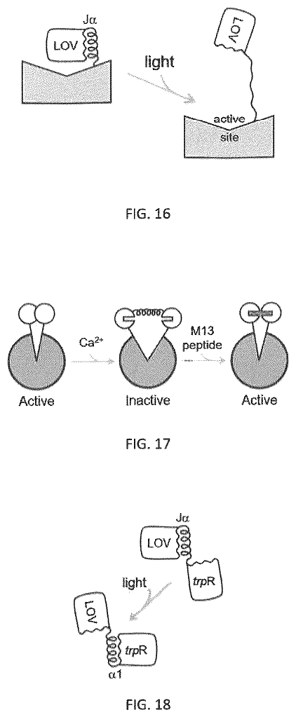

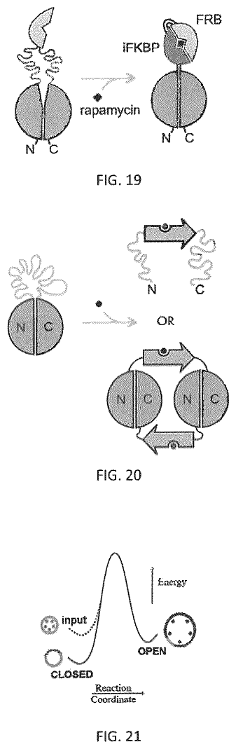

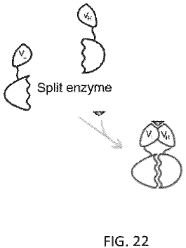

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a system for responding to the presence of an antigen or hapten analyte 100. The inventive system 100 utilizes protein engineering to create an IF-THEN coupling between an antibody 120 (input module), and a naturally switchable multisubunit protein (output module), which may be referred to as an allosteric multi-subunit protein 110. Naturally switchable proteins, which are technically referred to as being ‘allosteric’, can readily adopt either of two or more distinct conformational states (i.e. having different 3-dimensional shape and/or dynamics). These alternate conformational states will also have a significant discrepancy in their biological activity. Therefore, shifting between the two states will be associated with switching from a biological activity associated with a first state and a different, often opposite, biological activity associated with a second state. Due to these characteristics, allosteric multi-subunit proteins 110 may be used as a switch and effectively be turned CON′ or ‘OFF’ depending upon the conformational state of the protein 110. By way of example, and not as a limitation, the biological activity associated with a conformation state may be a level of enzyme activity, a membrane channel opening size, or the ability to bind to other proteins.

By way of example and not as limitations, natural switching between these alternative conformational states may be triggered by interaction with a regulatory ligand/protein, covalent modification such as Ser/Thr/Tyr phosphorylation, or other change in surrounding environment such as pH or transmembrane voltage. Many naturally allosteric multi-subunit proteins 110 are composed of multiple subunits, which are separate polypeptide chains that fold more-or-less independently and then associate predominantly through noncovalent interactions, which may sometimes be combined with disulfide bonds between Cys residues, to form the final assembled structure. In an amino acid sequence one or more of these subunits are often identical.

Antibody 120 for these designs refers to the variable superdomain, or Fv fragment, which is composed of two protein subunits. These subunits are the heavy chain variable domain subunit 130 and light chain variable domain subunit 140, commonly abbreviated as VH 130 and VL 140 respectively. This antibody 120 is solely responsible for recognition and binding to the target antigen or hapten (i.e. small molecule capable of eliciting an immune response directed toward it only when conjugated to a protein antigen carrier). When allosterically coupled, antigen or hapten binding to the Fv superdomain will cause a shift in the balance between alternate conformations of the naturally switchable ‘output’ protein, thereby eliciting a discernable change in enzyme activity, binding activity, ion conductivity, or other function of the ion. Consequently, presence of the antigen or hapten analyte can readily be detected through its effect on activity of the output or can be used to turn on the output for downstream functionality, such as drug delivery through the opened pore of a liposomal vesicle for example (vide infra).

In the absence of bound antigen or hapten, antibodies exhibit significant flexibility in the relative orientation of their VH 130 and VL 140 domains, however when ligand is bound this configuration becomes more rigid to provide an optimal combining site fit for its particular geometry and chemistry (vide infra). Consequently, at least some sites on the paired variable domains (including their C-termini) will be capable of adopting a range of separation distances when the antibody 120 is not complexed with its target antigen or hapten, but must adopt a more-or-less fixed position in the bound state. Meanwhile, shifts between alternate conformations of a naturally allosteric multi-subunit protein 110 are typically accompanied by changes in the relative spacing of points on the surface of its adjacent subunits. Furthermore, these shifts in 3-dimensional shape are generally accompanied by changes in dynamics of the allosteric multi-subunit protein 110, with one conformational state exhibiting substantially more flexibility than the alternate conformer. The inventive system utilizes covalent joining of VH 130 and VL 140 subunits of the antibody Fv 120 to separate subunits of the naturally allosteric multi-subunit protein 110 such that antigen or hapten binding induces a shift in the balance between alternate conformers of the latter, and therein controls functional activity (on/off, open/closed etc.) of this ‘output module’.

The inventive system 100 can be achieved by creating an incompatibility between attachment point separation of the antibody Fv 120 and those on the output module allosteric multi-subunit protein 110 subunits in its ‘expanded’ conformation, when the former is bound to its antigen or hapten but not when the target analyte is absent. This is due to flexing of VH 130 and VL 140 orientation of the unliganded Fv that allows it to stretch to accommodate larger separation of its attachment points in the expanded conformer of the naturally switchable allosteric multi-subunit protein 110 module. Alternatively, attachment points on the antibody Fv 120 and naturally switchable output allosteric multi-subunit protein 110 can be chosen to create a conflict between the distance separating those of the ‘condensed’ output protein conformer and that on the antibody Fv 120 rigidified by antigen or hapten binding. It is also possible to utilize dynamic ‘freezing’ of the antibody Fv 120 associated with ligand binding to stabilize the low flexibility conformer of the naturally switchable output protein through structural matching of the fixed attachment points and/or a rigid-to-rigid dynamic coupling.

In one embodiment, there may be an allosteric coupling of antibody Fv 120 input module to the MthK potassium channel as output via fusion of its V-domains (two of each) to the four identical membrane-distal RCK subunits of the MthK cytoplasmic gating-ring octamer. By allosterically coupling antigen or hapten binding to modulate balance between alternate expanded or contracted conformers of the MthK gating-ring, a powerful biosensor may be constructed. Membrane channel 150 opening triggered by the expanded gating-ring conformer may facilitate passage of potassium ions through the membrane 160, and thus provide an electrical conductivity gate. In such an embodiment, a signal commensurate with antigen binding may be measured using a simple conductivity reader, which may be smartphone based.

Molecular electrophysiological studies of the allosteric multi-subunit protein 110 of the inventive system 100 and other potassium channel proteins yield conductivity measurements from an individual molecule of the channel protein. This not only provides an extremely inexpensive device construction, given the miniscule quantities of engineered channel protein required, but also yields a high level of sensitivity because the binding of a single molecule of analyte compound to an individual channel receptor may be monitored. Given the standalone capability and direct electrical interface of the design, this device may be utilized to guide internally circulating nanorobotic ‘doctors’ of the future. By way of example, and not as a limitation, this may be accomplished by automatically eliciting programmed medical response to biological markers of disease.

The inventive system 100 is a design format that enables one skilled in the art to allosterically couple any antibody 120 to any naturally allosteric multi-subunit protein 110. The inventive system 120 may be applied to make any of a plethora of possible output functionalities be responsive to any chosen antigen or hapten. This provides for a wide range of individual molecular devices. By way of example, and not as a limitation, there may be molecular designs for a series of constructs that allosterically couple antibody Fv 120 to the bacterial wide-bore membrane channel, MscL. This channel is a homopentamer that normally adopts a closed-channel configuration, but when triggered by membrane stretch resulting from elevated intracellular osmotic pressure, which, by way of example, and not as a limitation, may be associated with microbial exposure to rainwater, will readily adopt an expanded or ‘open’ conformation that creates a ˜3 nm pore through the cell membrane 160. This may protect the organism from being lysed under these conditions by releasing cellular contents including even small proteins. When placed in the membrane 160 of a drug-loaded liposome, such a device may facilitate gated release of virtually any compound contained within. This has may allow for targeted drug delivery via an allosteric coupling of MscL to antibody Fv 120, which is specific for a particular biomarker of disease or specific for a harmful physiological event such as epileptic seizure or excessive inflammation.

By way of example, and not as a limitation, one embodiment provided herein may use direct head-to-tail fusion of the antibody 120 variable domains to the MthK gating-ring subunits, with C-terminus of the VH 130 and VL 140 subunits being linked to N-termini of the membrane distal RCK subunits, via a Glyn-Ser peptide. The fused polypeptides are encoded in single long open reading frames having upstream antibody variable, and downstream RCK, regions. RCK subunit N-terminus used for current designs is the first residue visible in the X-ray structure, but alternate junction choices, including the natural N-terminus of RCK, may also be utilized. Additionally, amino acid sequence and composition of the linker itself may further improve efficacy of the design. Use of circularly permuted versions of the antibody 120 variable domains and/or RCK subunit polypeptides, provide the option to exploit different attachment points for the linker-peptide mediated joining of these modules. Alternate design configurations may be achieved through use of site-directed chemical conjugation. Finally, optimization of initial leads through mutagenesis (semi-rational and/or non-biased) and screening is a fundamental part of the allosteric engineering process, and we this is true for the inventive system 100 as well. Those skilled in the art will appreciate that current state-of-the-art in allosteric multi-subunit protein 110 design has optimizations and contingencies, such as those discussed above.

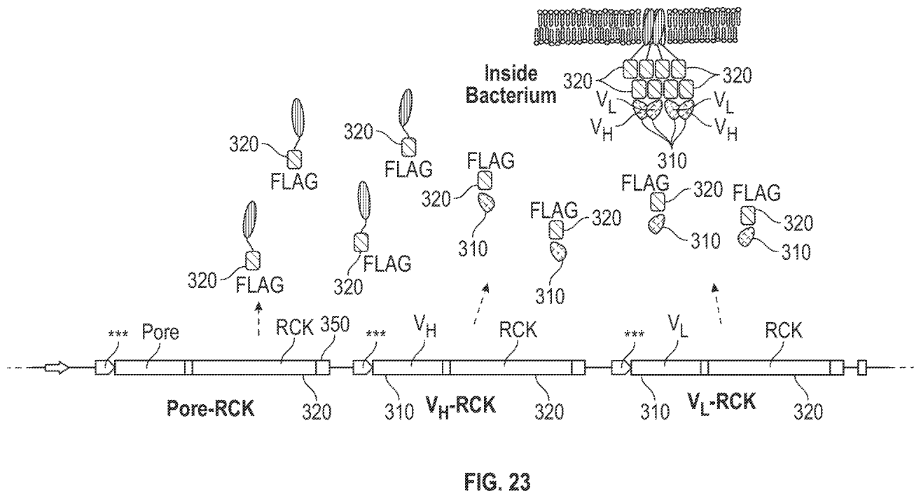

One embodiment of this invention is a fusion of the antibody heavy and light chains to the membrane distal RCK subunits of the cytoplasmic gating-ring of the archaebacterial potassium channel, MthK, which may be referred to as MthK-Fv′ This MthK-Fv ion channel protein may be produced using a tri-cistronic gene in which coding regions for three polypeptides, each having the RCK subunit coding region of MthK linked head-to-tail to the coding regions of the MthK protein transmembrane domain (Pore-RCK), in one case, and to the light and heavy chain antibody variable domain sequences for the other two, i.e. VL-RCK and VH-RCK, respectively (see below). Transcription of the gene could be driven by the very powerful and specific T7 DNA polymerase, which in a host strain has been found useful, which is produced from a chromosomal gene under control of the IPTG-inducer. Moreover, this host strain is tailored for intracellular expression of normally secreted extracellular proteins that contain cystine disulfide bonds and may be helpful for producing MthK-Fv since antibody Fv are disulfide-containing domains and the gating ring to which they are fused, is cytoplasmic. The light and heavy variable domains in this embodiment can be from an antibody such as 4D5Flu, which has specificity for the fluorescein hapten, with the fusions for MthK-Fv including residues Asp1-Ala109 and Glu1-Ser113 of its light and heavy chains, respectively (Kabat numbering). The RCK subunit may comprise sequence starting at residue His117 of the full length MthK polypeptide and continuing to the natural C-terminus, with a Gly4-His6 C-terminal tag added to Pore-RCK to aid purification and immobilization of the channel protein (see below). The antibody variable domain C-termini (for light and heavy chains) may be connected to the N-terminus of RCK through ‘linker’ peptide regions that may be optimized through testing a range of linker lengths and generally Gly-rich, sequences. We have found it possible to express MthK-Fv with linkers for the light and heavy variable domain to RCK fusions being as short as 4 residues, and up to 15 amino acids in length, but have not tested beyond this range. When transformed into a host E. coli strain such as SHuffle T7 Express, this gene directs expression of three polypeptides that assemble into the octameric MthK-Fv channel protein, as depicted in FIG. 23. It may be beneficial to conduct expression at reduced growth incubator temperature (eg. 18° C.) and for extended periods (20-24 hrs) with a relatively low (20 mM) concentration of IPTG inducer.

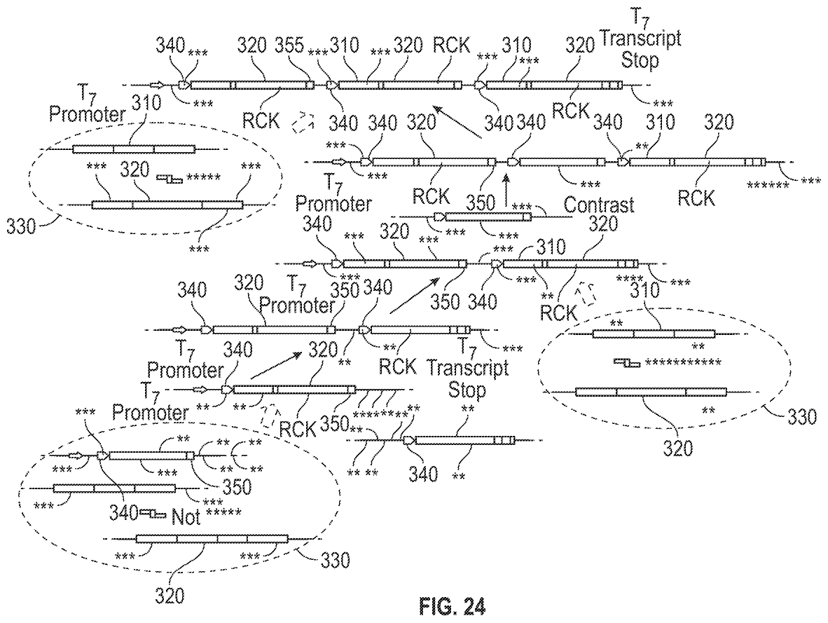

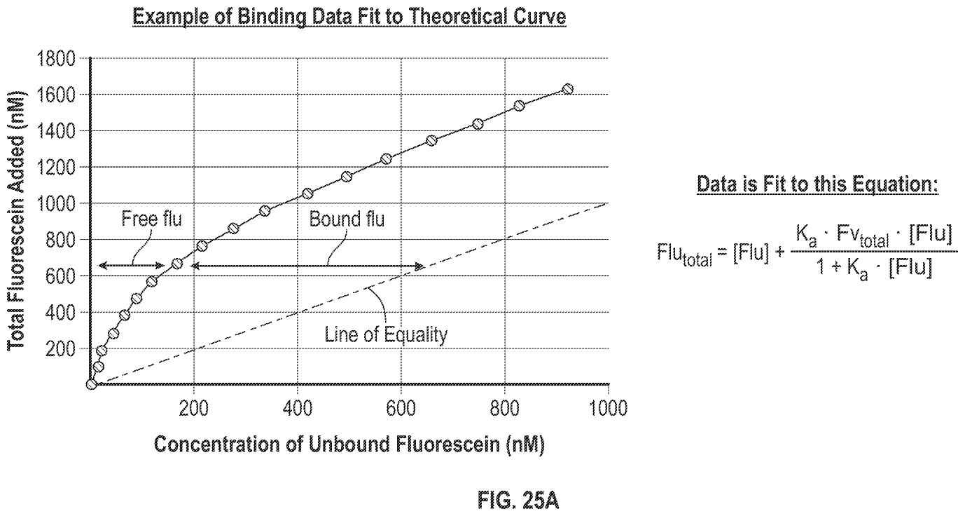

Recombinant DNA for ‘MthK-Fv’ genes having a range of sequences for linkers between the light or heavy variable domain (of antibody Fv, depicted in FIGS. 23 and 24 as 310) and the RCK subunit (depicted in FIGS. 23 and 24 as 320), may be constructed with synthetic oligonucleotides encoding the linker (light blue), combined with plasmids containing (custom synthetic) DNA encoding each of the variable domains and RCK, in a 16° C./37° C. thermocycler reaction (‘IIs/Ligase’ r×n; similar to Golden Gate Assembly—New England Biolabs) that utilizes a Type-IIs restriction endonuclease (such as Bpil or Sapl) and T4 DNA ligase (330 as depicted in FIG. 24). Meanwhile DNA encoding ‘Pore-RCK’ is similarly constructed using plasm id carrying the MthK transmembrane domain (Pore) combined with the one for RCK, and a synthetic linker corresponding to the natural amino acid sequence connecting Pore and RCK in the full-length MthK polypeptide. The linker-encoding DNA fragments can be efficiently produced from oligonucleotides having complementarity in the 3′ regions using a high-fidelity thermostable DNA polymerase such as Q5 from New England Biolabs. Acceptor plasmid for each of the Ils/ligase assembly reactions is created by conventional cloning of custom DNA containing the ribosome binding site (rbs) for translation of each of the MthK-Fv polypeptides and Bpil restriction sites, sequentially into a high-copy T7-promoter containing plasmid such as pGEM (we found it helpful to remove Lac operator sequence from the original pGEM vector). Tripartite assembly of coding regions into acceptor vectors with the flanking RBS 340 and tag/stop codon (His6 350) regions can be done one at a time, with the middle, i.e. 2nd, coding region inserted last. Complete control of sequence of the non-coding DNA, including the RBS segment which dictates how much of each polypeptide is expressed, is achieved by using a nested IIs/Ligase approach with Sapl, to seamlessly insert this acceptor between the assembled first and third coding regions as depicted in FIG. 24. We have found RBS sequences designed using the www.denovoDNA.com tool to be effective, particularly if strengths for translation of the Pore-RCK (first), VH-RCK (second) and VL-RCK (third) coding regions are set to roughly 8K, 80K and 12K, respectively.

MthK-Fv protein can be extracted from frozen cell pellets of expression culture by resuspending in ice cold HEPES buffer (40 mM, pH 7.0; 1.5 ml for 30 ml of culture) containing lysozyme and DNAse I with two additional cycles of snap-freezing and thawing to lyse the cells, and then using decyl maltoside (50 mM in presence of 100 mM KCl with end-over-end mixing for 6 hrs) to solubilize the membrane channel protein. A His6-tag encoded at the C-terminus of the Pore-RCK (i.e. natural full-length MthK, first coding region in FIGS. 23 and 24) polypeptide can facilitate capture of the assembled MthK-Fv octameric channel protein on an immobilized metal affinity chromatography (IMAC) beaded resin such as TALON Superflow (TaKaRa Biosciences). Immobilization of at least some portion of the detergent-solubilized MthK-Fv protein in the cleared (by centrifugation at 20K×g at 4° C. for 45 min) extract onto a small volume of IMAC beads (eg. 60 ml per 30 ml of culture) was possible by mixing on a bottle roller for 10-12 hours at 4° C. (with 3-fold diluted extract). After washing away unbound material through repeated steps whereby the beads are separated from the liquid phase (either by gravity, or by spinning at 700×g for 3 min at 23° C.) and then resuspended in fresh buffer, fluorescein-binding activity could be measured using a bead-based titration assay (see below). Fluorescein-binding detected in this way is dependent upon assembly of the octameric MthK gating-ring and its associated membrane channel, as immobilization on the IMAC beads is facilitated by His6-tag attached exclusively to the Pore-RCK subunit while fluorescein-binding is of course, mediated by the VL and VH-RCK polypeptides. Moreover, elution of immobilized material from the IMAC beads following the titration assay yielded just two visible bands by Coomassie-stained SDS-PAGE electrophoresis; estimated molecular weights for these were consistent with assignment as VL and VH-RCK (very similar sizes) for the lower, and an expected Pore-RCK tetramer, for the upper band.

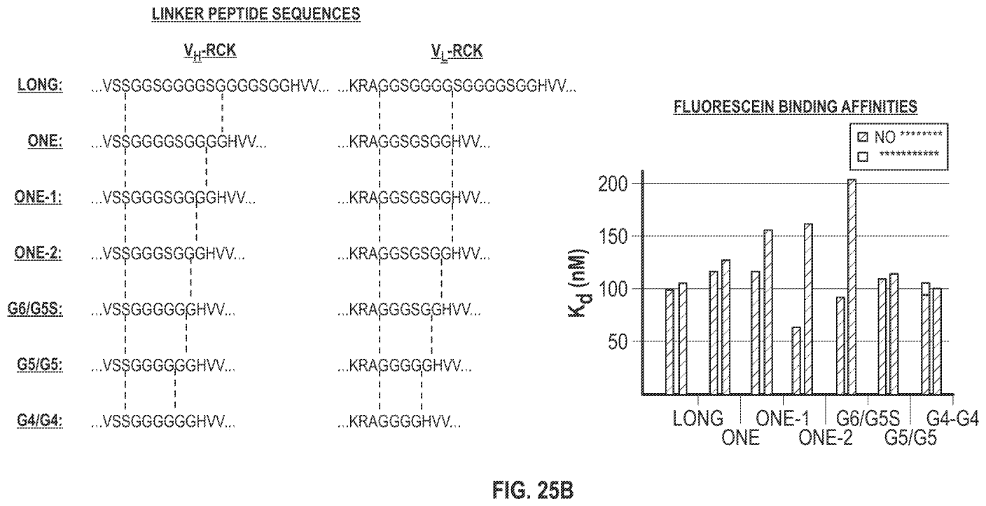

Fluorescein-binding activity measured in the bead-based titration assay can be used to screen gene constructs (particularly different RBS strengths) and growth conditions for efficient expression of the MthK-Fv channel protein, as well as test for allosteric coupling of the gating-ring expansion and channel opening, with hapten-binding by the antibody Fv portion of the fusion protein. This assay involves stepwise addition of known amount of fluorescein with fraction remaining unbound to the immobilized MthK-Fv being determined each time using spectrophotometric measurement (at 490 nm) after the IMAC beads have been allowed to sink to the bottom of the cuvette and out of the light path. Binding curves generated from the collected data can be fit to a theoretical equation (see FIG. 25A) that then provides measurement of the total amount of fluorescein-binding activity (Fvtotal), as well as affinity of the Fv for its fluorescein hapten (Ka). By conducting the assay in the absence vs. presence of calcium chloride (30 mM), it is possible to compare fluorescein affinity of the MthK-Fv protein with its gating-ring in the contracted (i.e. membrane channel closed) vs. expanded (channel open) conformational states, respectively. Discrepancy between fluorescein affinities obtained under these conditions is direct evidence that activity of the antibody Fv of the fusion protein is responsive to gating-ring expansion and channel opening of its MthK part. In other words, channel opening and hapten-binding are allosterically coupled, which means that channel opening/closure of the MthK-Fv is also responsive to hapten-binding by its antibody Fv portion. Therefore, by assessing fluorescein-binding affinities of MthK-Fv with various linker lengths and sequences (for VL and VH-RCK), it is possible to predict which of the screened linker configurations can be expected to provide fusions in which channel opening is responsive to hapten-binding by the Fv.

Data summarized in FIG. 25B provides an example wherein MthK-Fv linkers of various lengths were screened for discrepancies in fluorescein-binding affinity in the presence vs. absence of calcium chloride (30 mM). This method provides a quick means to evaluate which of the different linker, fusion protein constructs, are allosterically coupled, that is to say, which of them will exhibit channel opening/closure in response to binding of the fluorescein hapten by the Fv. As expected, our fusions with long linkers connecting antibody Fv to the membrane-distal ring of RCK subunits (eg. LONG and ONE) exhibit no significant change in fluorescein affinity because these linkers absorb Ca2+-induced expansion of the gating-ring structure, without impacting the Fv. Short linkers (eg. G5/G5 and G4/G4) on the other hand, also yield essentially no change in fluorescein affinity with vs. without calcium chloride present, and this could be because we've reached the point where their short length prevents gating-ring expansion without also causing physical separation of the light and heavy chain variable domains. Since the energetic cost of doing so is very high, this would likely block gating-ring expansion altogether, and presumably Ca2+ binding as well. Meanwhile, length of intermediate sized linkers (eg. ONE-2 and G6/G5S) is such that gating-ring expansion can be accommodated by a distortion of the antibody Fv structure, particularly in its relative orientation of the light and heavy chain variable domains. This energetically less costly accommodation presumably allows gating-ring mediated expansion to be allosterically coupled to the antibody Fv, as observed in the 2.6 and 2.2-fold changes in fluorescein-binding affinity in response to calcium for ONE-2 and G6/G5S, respectively, as depicted in FIGS. 25A and 25B. Existence of this allosteric window is a direct result of antibody flexibility, presumably in relative orientation of the VL and VH domains, because it allows the Fv to distort to accommodate gating-ring expansion without completely disrupting its structural integrity. Moreover, allosteric coupling of these functions means that it should be possible to use fluorescein binding to control channel opening/closing of these MthK-Fv proteins and thus changes in electroconductivity across a membrane bilayer containing these MthK-Fv fusion proteins could be used to detect presence of the hapten.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

What is claimed is:

1. A system for responding to the presence of an antigen or hapten analyte comprising:
an allosteric multi-subunit protein having a first conformational state associated with a first biological activity and a second conformational state associated with a second biological activity; and
an antibody comprising:
a heavy chain variable domain subunit of the allosteric protein fused to a first subunit of the allosteric multi-subunit protein, and
a light chain variable domain subunit of the allosteric protein fused to a second subunit of the allosteric multi-subunit protein;
wherein the first biological activity is observable when the antigen or hapten analyte is bound to the heavy chain variable domain subunit and the light chain variable domain subunit; and
wherein the second biological activity is observable when the antigen or hapten analyte is not bound to the heavy chain variable domain subunit and the light chain variable domain subunit.

2. The system of claim 1 wherein the allosteric multi-subunit protein is fused to the heavy chain variable domain subunit with a linker polypeptide; and
wherein the allosteric multi-subunit protein is fused to the light chain variable domain subunit with a linker polypeptide.

3. The system of claim 2 wherein the heavy chain variable domain subunit is fused to a first amino-terminal end of the allosteric multi-subunit protein and the light chain variable domain subunit is fused to a second amino-terminal end of the allosteric multi-subunit protein.

4. The system of claim 3 wherein the heavy chain variable domain subunit is fused head to tail with a first naturally occurring end terminus of the allosteric multi-subunit protein.

5. The system of claim 3 wherein the light chain variable domain subunit is fused head to tail with a second naturally occurring end terminus of the allosteric multi-subunit protein.

6. The system of claim 3 wherein the heavy chain variable domain subunit is fused head to tail with a first naturally occurring end terminus of the allosteric multi-subunit protein; and
wherein at least a portion of a sequence of the allosteric multi-subunit protein is removed.

7. The system of claim 3 wherein the light chain variable domain subunit is fused head to tail with a second naturally occurring end terminus of the allosteric multi-subunit protein; and
wherein at least a portion of a sequence of the allosteric multi-subunit protein is removed.

8. The system of claim 3 wherein the heavy chain variable domain subunit is fused head to tail with a new end terminus of the allosteric multi-subunit protein created by a rearrangement of a gene coding region.

9. The system of claim 3 wherein the light chain variable domain subunit is fused head to tail with a new end terminus of the allosteric multi-subunit protein created by a rearrangement of a gene coding region.

10. The system of claim 1 wherein the first biological activity is an enzyme activity having a first level and the second biological activity is the enzyme activity having a second level different than the first level.

11. The system of claim 1 wherein the first biological activity is a membrane channel opening having a first permeability and the second biological activity is the membrane channel opening having a second permeability different than the first permeability.

12. The system of claim 8 wherein the membrane channel further comprises a domain residing external to a membrane wherein the domain affects the permeability of the membrane channel.

13. The system of claim 9 wherein the allosteric multi-subunit protein comprises:
MthK, and wherein the MthK further comprises:
cytoplasmic gating-ring octamer.

14. The system of claim 10 wherein the first biological activity and the second biological activity are observable by detecting a change in permeability by measuring electrical conductivity caused by a passage of a plurality of potassium ions through the membrane channel.

15. The system of claim 8 wherein the membrane channel does not include a domain external to a membrane.

16. The system of claim 1 wherein the first biological activity protein binding affinity having a first level and the second biological activity is the protein binding affinity having a second level different than the first level.
